SEQUENCE LISTING (1 sequence)

SEQ ID NO: 1 - protein, 84 aa, Homo sapiens
SVSEIQLMHNLGKHLNSMERVEWLRKKLQDVHNFVALGAPLAPRDAGSQRPRKKEDNVLVESHEKSLGEADKADVNVLTKAKSQ

(19) United States
(12) Patent Application Publication
(10) Pub. No.: US 2015/0238574 A1
(43) Pub. Date: Aug. 27, 2015

(54) METHOD FOR TREATING BONE LOSS USING PARATHYROID HORMONE

(71) Applicant: NPS Pharmaceuticals, Inc., Bedminster, NJ (US)

(72) Inventors: David Wells, Salt Lake City, UT (US); John Fox, Salt Lake City, UT (US)

(21) Appl. No.: 14/575,944

(22) Filed: Dec. 18, 2014

Related U.S. Application Data
(63) Continuation of application No. 10/986,000, filed on Nov. 12, 2004, now abandoned.
(60) Provisional application No. 60/613,508, filed on Sep. 28, 2004.
(60) Provisional application No. 60/523,116, filed on Nov. 19, 2003.
(60) Provisional application No. 60/518,871, filed on Nov. 12, 2003.

(51) Int. Cl. A61K 38/29; A61K 45/06; A61K 9/19

(52) U.S. Cl. CPC A61K 38/29; A61K 9/19; A61K 45/06

(57) ABSTRACT

The present invention relates to method for treating bone loss utilizing full length parathyroid hormone. The method dose not increase the risk of osteosarcoma or bone lesions.

### CROSS REFERENCE TO RELATED APPLICATIONS

This application claims the benefit of priority to U.S. provisional application No. 60/518,871, filed Nov. 12, 2003, U.S. provisional application No. 60/523,116, filed Nov. 19, 2003, and U.S. provisional application No. 60/613,508, filed Sep. 28, 2004, all of which are herein incorporated by reference in their entirety.

### FIELD OF THE INVENTION

The present invention relates to a method for treating bone loss with safe dosages of full-length parathyroid hormone.

### BACKGROUND OF THE INVENTION

I. Background Regarding Parathyroid Hormone

Parathyroid hormone (“PTH”) is an 84-amino acid peptide produced and secreted by the parathyroid gland to regulate bone and mineral homeostasis. The initial protein product produced by the gland, “preproPTH,” is a 115-amino acid peptide. The 31 amino acids at the N-terminus of the peptide are important for transporting the peptide into the endoplasmic reticulum. PreproPTH is then hydrolyzed to “proPTH,” which possesses 6 amino acids at its N-terminus that are ultimately cleaved to form the active hormone, namely “PTH(1-84).”

It is known that the first 34 N-terminal amino acids have the same activation as the full length PTH(1-84) at the only known receptor for PTH, “PTH1R”. See, for instance, Potts et al., Amer. Journ. of Med., 50: 639-649 (1971); Potts et al., Proceed. of the 3rd Intern. Symp. of Endocrin., London, pp 333-349 (1971); Tregear et al., Endocrin., 93: 1349-1353 (1973). In addition, it has been shown that C-terminal fragments, such as 39-84 and 53-84, do not compete with the 1-34 fragment for receptor binding. Furthermore, these C-terminal fragments did not activate adenylate cyclase, all of which led to the conclusion that the C-terminal portion of the PTH peptide was irrelevant. See, Segre et al., Journ. of Bio. Chem., 254: 6980-6986 (1979); Nissenson et al., Journ. of Bio. Chem., 254: 1469-1475 (1979); Potts et al., Adv. in Prot. Chem., 35: 323-396 (1982).

Amino acids 1 and 2 of the N-terminal end of PTH facilitate the anabolic effects of PTH-1 receptor activation, which manifests in bone growth. Among other responses, PTH-1 receptor activation leads to an increase in osteoblast numbers and function, which occurs by activation of existing osteoblasts, stimulation of osteoblast formation from precursor cells by activation of bone lining cells, and inhibition of osteoblast apoptosis. Together, these lead to an increased rate of new bone formation.

However, even though the C-terminal end of PTH was deemed to not be relevant to biological activity, it has been found that the C-terminal portion of the peptide is necessary for normal transport and processing. See, for instance, Kemper et al., Proceed of the Nat. Acad. of Sciences, 71: 3731-3735 (1974); Freeman et al., Molec. Endocrin., 1: 628-638 (1987); Wiren et al., Journ. of Bio. Chem., 263: 19771-19777 (1988); Cioffi et al., Journ. of Bio. Chem., 264: 15052-15058 (1989); Karaplis et al., Journ. of Bio. Chem., 270: 1629-1635 (1995); Lim et al., Endocrin., 131: 2325-2330 (1992). In particular, there is evidence that full-length PTH is cleaved to C-terminal fragments within the parathyroid gland, and that these fragments are secreted in response to increased calcium ion concentrations in the blood. There is no evidence to date that N-terminal fragments are stored within or secreted from the gland except in the form of intact PTH. See Habener et al., Nature-New Biol., 238: 152-154 (1972); Flueck et al., Journ. of Clin. Invest., 60: 1367-1375 (1977); Mayer et al., Endocrin.,104: 1778-1784 (1979); Chu et al., Endocrin., 93: 915-924 (1973); Habener et al., Endocrin., 97: 431-441 (1975); Russell et al., Journ. of Clin. Invest., 72: 1851-1855 (1983); Heinrich et al., Endocrin., 112: 449-458 (1983); Brookman et al., Journ. of Bone & Min. Res., 1: 529-537 (1986); Sherwood et al., Proceed. of the Nat. Acad. of Sciences, 67: 1631-1638 (1970); Arnaud et al., Amer. Journ. of Med., 50: 630-638 (1971); Hanley et al., Journ. of Clin. Invest., 62: 1247-1254 (1978); DiBella et al., Journ. of Clin. Endocrin. & Metab., 46: 604-612 (1978); Roos et al., Journ. of Clin. Endocrin. & Metab., 53: 709-721 (1981); MacGregor et al., Endocrin., 112: 1019-1025 (1983); Hanley et al., Journ. of Clin. Endocrin. & Metab., 63: 1075-1079 (1986); Morrissey et al., Endocrin., 107: 164-171(1980); MacGregor et al., Journ. of Biol. Chem., 261: 1929-1934 (1986); MacGregor et al., Journ. of Biol. Chem., 254: 4428-4433 (1979); Kubler et al., Experim. & Clin. Endocrin., 88: 101-108 (1986); Schachter et al., Surgery, 110: 1048-1052 (1991); Tanguay et al., Endocrin., 128: 1863-1868 (1991).

Furthermore, it is now apparent that the C-terminal region of PTH has a novel receptor which is specific for this region of the hormone. See, for instance, Hodsman et al., J. Clin. Endocrinol. Metab., 88, pp. 5212-5220 (2003). Accordingly, the full-length PTH has biological properties that are distinct from those of N-terminal PTH analogs.

However, the importance and effects of full length parathyroid hormone on bone growth, calcium physiology, and replenishment are still not readily understood as, maybe, for example, the effects of calcium. The normal daily rise and fall of PTH levels in the blood have a profound effect on bone, and injections of PTH can stimulate the growth of new bone in cases where bone has been lost to osteoporosis.

II. Background Regarding Osteoporosis

Osteoporosis is estimated to affect one half of all women over 50 years of age in the U.S. Approximately 10 million women and 2 million men in the U.S. have advanced osteoporosis and another 18 million are at high risk of fractures because of low bone mass. Osteoporosis is responsible for 1.5 million fractures annually in the U.S. The estimated direct national expenditures for treating osteoporosis and its consequences are nearly $14 billion each year. Overall, the fatality rate for hip fracture patients within one year of the fracture is 24%, and survivors are often institutionalized as a result of disability. Researchers at the Fourth International Symposium on Osteoporosis, which convened in June 1997, estimated that by the year 2015 osteoporosis could affect nearly 41 million Americans.

Current therapies for osteoporosis include administering supplemental dietary calcium and vitamin D to help slow the rate of bone loss. In postmenopausal women, hormone replacement therapy with, for example, estrogen, decreases the rate of bone resorption, but does not reverse the loss of bone mass. Other therapies include the use of compounds such as bisphosphonates and raloxifene. Such drugs can halt bone loss and have shown modest bone building effects in some studies.

Studies in humans with various fragments of PTH and full length PTH have demonstrated an anabolic effect on bone, and have prompted significant interest in the use of PTH for the treatment of osteoporosis and related bone disorders. The mechanism by which bone is constantly renewed is called bone remodeling. PTH acts on the bone remodeling process so that new bone is generated and added to the skeleton faster than old bone is broken down. This anabolic action occurs when PTH is administered and is in contrast to current osteoporosis treatments that only work to slow or stop bone loss. The significant anabolic effects of PTH on bone, including stimulation of bone formation, which results in a net gain in bone mass and/or strength, have been demonstrated in many animal models and in humans.

In a one-year trial in 174 postmenopausal women, daily injections of 50 to 100 micrograms of recombinant human PTH(1-84) produced an average increase in bone mineral density of the spine of 8% (see U.S. Pat. No. 6,284,730). Also, a one year study was conducted in 119 postmenopausal women who received 100 μg full-length PTH daily. This study also showed an increase in bone mineral density in the spine (Black et al., NEJM, Vol. 349, No. 13, p. 1207, 2003).

In December of 2002, an N-terminal PTH(1-34) teriparatide product, Forteo™ (Eli Lilly & Co.), was approved by the U.S. Food and Drug Administration for treatment of osteoporosis. However, it was surprisingly found that Forteo™, which consists of the biologically active PTH(1-34) N-terminus, when administered to rats resulted in a dramatic increase in the incidence of osteosarcoma, as well as other bone lesions (Vahle et al., Toxicologic Pathology, Vol. 35, No. 3, p. 312 (2002)). Indeed, it was reported that the “relative risk” of osteosarcoma in rats treated with PTH(1-34) was 29-times greater when rats were dosed at 5 μg/kg/day, 138-times greater in rats given 30 μg/kg/day, and 225-times greater in rats given 75 μg/kg/day, assuming a background incidence of osteosarcoma of 0.2% (Kuijpers G., Endocrinologic and Metabolic Drugs Advisory Committee Meeting, Bethesda, Md., Jul. 27, 2001). The exposure in humans with a clinical dose of PTH(1-34) of 20 μg/day is about 1/3 the exposure in the lowest dose tested in rats. There was no dose of PTH(1-34) which was tested in this two year rat study which did not result in an increased risk for osteosarcoma and bone lesions. Even so, Forteo™, is the only FDA-approved and commercially-available PTH product. Although the FDA stated that the clinical relevance of the rat bone neoplasms induced by teriparatide (PTH(1-34)) is unclear, the FDA concluded that there is a potential increase in the risk for bone neoplasms in humans treated with teriparatide.

Osteosarcoma is a type of bone cancer that develops in the osteoblast cells that form the outer covering of bone. It most often occurs in children, adolescents, and young adults. Approximately 900 new cases of osteosarcoma are reported each year in the US. It occurs nearly twice as often in males, and represents 5 percent of all childhood cancers. Osteosarcoma may metastasize, or spread, into nearby tissues of the foot, or into tendons or muscles. It may also metastasize through the bloodstream to other organs or bones in the body.

When tied to the increased chance of developing osteosarcoma, administering PTH(1-34) to a human is not a desirable therapeutic option, particularly to subjects suffering, or at risk of developing, osteosarcoma. As the PTH(1-34) fragment is the only currently approved PTH product for treating osteoporosis which results in an increased bone mass, there is an urgent need in the art for improved safer treatments for bone related diseases wherein increases in bone mass are desirable.

### SUMMARY OF THE INVENTION

The present invention satisfies the need for a safer bone building product by providing a full-length, intact, PTH, i.e., PTH(1-84) composition at therapeutically effective and safe dosages that unexpectedly and surprisingly do not result in significant abnormal bone growth, osteosarcoma, or bone lesions.

In addition, the present invention satisfies the need for a bone building product which can be safely administered to patient populations which may not tolerate other PTH formulations, such as PTH(1-34).

More particularly, in one aspect of the present invention, a method for treating a subject suffering from, or at risk of, bone loss is provided. This method comprises administering to the subject a composition comprising a dosage of full-length parathyroid hormone (PTH(1-84)), wherein the dosage does not exceed about 600 μg/person/day, wherein the subject is selected from a patient population suffering from, or at risk of, developing osteosarcoma.

In another aspect, another method for treating a subject suffering from, or at risk of, bone loss is provided, which comprises administering to the subject a composition comprising a dosage of full-length parathyroid hormone (PTH(1-84)), wherein the dosage does not exceed about 600 μg/person/day, wherein the subject is selected from a patient population which do not take PTH(1-34) because of the increased risk of osteosarcoma associated with PTH(1-34) in rats.

In yet another aspect, a method for reducing the risk of osteosarcoma induced by an N-terminal PTH fragment in a subject is provided, which comprises administering to the subject a pharmaceutical composition comprising intact parathyroid hormone (PTH(1-84)).

A further aspect of the present invention entails a method for reducing the risk of osteosarcoma induced by an N-terminal PTH fragment in a subject, comprising administering to the subject a pharmaceutical composition comprising a C-terminal fragment of parathyroid hormone.

One other aspect of the present invention encompasses a method for treating a subject suffering from, or at risk of, bone loss comprising administering to the subject a pharmaceutical composition comprising full-length parathyroid hormone (PTH (1-84), wherein (a) such administration does not result in a significant increased risk of osteosarcoma; and (b) the subject is selected from a patient population which do not take PTH(1-34) because of the increased risk of osteosarcoma associated with PTH(1-34) in rats.

The decreased risk of osteosarcoma established herein for PTH(1-84) may allow certain patient populations to be treated who are otherwise suffering from, or at risk of osteoporosis, Such potential populations include: (1) subjects with Paget's disease of bone; (2) subjects with elevated levels of alkaline phosphatase; (3) pediatric subjects or young adults with open epiphyses; (4) subjects having a prior history of radiation therapy involving the skeleton; (5) subjects with bone metastases or a history of skeletal malignancies; and (6) subjects with metabolic bone diseases other than osteoporosis.

In one other aspect of the present invention, a package is provided, which comprises one or more daily doses of 100 μg/day dose of full-length PTH(1-84) and written instructions, wherein the written instructions provide that the pharmaceutical composition is to be administered to a patient who is suffering from osteoporosis and wherein risk of increased osteosarcoma has not been seen in rats at dosages exhibiting about a 3 to 4 fold increase in exposure to PTH(1-84).

Both the foregoing general description and the following detailed description are exemplary and explanatory and are intended to provide further explanation of the invention as claimed. Other objects, advantages, and novel features will be readily apparent to those skilled in the art from the following detailed description of the invention.

### BRIEF DESCRIPTION OF THE DRAWINGS

FIG. 1 is a graph depicting the dose-response relationship between the AUCᵣₐₜ/AUCₕᵤₘₐₙ exposure ratio and incidence of osteosarcoma from male and female rats treated with PTH(1-84).

FIG. 2 shows graphs depicting dose related increases in bone mineral content (BMC). The graphs show that the mean±SD DXA BMC at the lumbar spine and central and distal regions of the femur was significantly increased for all PTH groups compared to combined controls groups after 24 months of treatment. *: p<0.001 (t-test), males and females on left and right panels, respectively.

FIGS. 3A and 3B are lateral views of whole-body radiographs. FIG. 3A: Control female. FIG. 3B: Female treated with PTH (1-84) at 150 μg/kg/day. The rat in FIG. 3B shows a generalized increased radiodensity of the skeleton with evident thickening of the calvarium, osteosclerosis of the long bones and obliteration of medullary spaces in femurs. In addition, focal bone losses in left proximal humerus (arrowhead) and in left proximal tibia (arrow) were subsequently diagnosed as multicentric osteosarcomas.

FIG. 4 shows gender-combined localization of primary osteosarcomas in rats treated with PTH (1-84).

FIGS. 5A-5F show common histological features of PTH (1-84)-induced neoplasms stained by hematoxilin and eosin (“H&E”). FIGS. 5A and 5D: Female given 150 μg/kg/day, osteoplastic osteosarcoma in thoracic vertebral body. The zonation phenomenon is evident with centrally located tumor bone (*) and anapestic osteopblasts at outer margin (arrow). FIGS. 5B and 5E: Female given 150 μg/kg/day, fibroblastic osteosarcoma in tibial proximal metaphysic (arrowhead). Radiographic evaluation, shown in FIG. 3B, was required to diagnose this neoplasm. FIGS. 5C and 5F: Male given 50 μg/kg/day, well defined osteoblastoma (arrows) in tibial proximal metaphysis.

FIGS. 6A and 6B show subgross histological sections of femoro-tibial joint. FIG. 6A: Control female. FIG. 6B: Female treated with PTH 150 μg/kg/day. Severe diffuse osteosclerosis with complete obliteration of the marrow cavity in femur, tibia and patella (arrow). H&E.

FIG. 7 shows a schematic diagram for the role of the C-terminal region of PTH (1-84).

FIG. 8 shows a comparative dose response curve for teriparatide and PTH (1-84).

### DETAILED DESCRIPTION OF THE PREFERRED EMBODIMENTS

The present invention provides safe dosages of full-length parathyroid hormone, i.e., PTH(1-84). Subjects in need of such treatment include, but are not limited to, subjects requiring increased bone mass, particularly subjects at risk of, or suffering from, bone loss, including bone loss from osteoporosis. More particularly, osteoporotic subjects at risk of developing, or suffering from, osteosarcoma are subjects in need of safe treatment for increasing bone mass.

While it was previously hypothesized that the C-terminal was not important to the biological activity of the PTH molecule in increasing bone mass, the present invention determines that the presence of the C-terminal portion of the PTH molecule results in a dramatically safer product for increasing bone mass as compared to N-terminal PTH (1-34).

Moreover, the present invention is directed to the surprising discovery that certain dosages of the full length PTH result in no, or a minimal, increased risk in osteosarcoma or bone lesions. This is contrary to what was expected. It was expected that full-length PTH which has a similar anabolic response as PTH (1-34), would also result in bone proliferative lesions (Vahle et al., “Skeletal Changes in Rats Given Daily Subcutaneous Injections of Recombinant Human Parathyroid Hormone (1-34) for 2 years and Relevance to Human Safety,” Toxicologic Pathology Vol. 35, No. 3, p. 312 (2002)).

The present invention is directed to the surprising discovery that there are effective, safe dosages of full length PTH (1-84), which comprises the regulatory C-terminus, which do not result in osteosarcoma or bone lesions. This is surprising given the published results regarding PTH(1-34), which produced a dramatic increased rate of osteosarcoma in rats even at the lowest dosage tested.

Indeed, certain categories of patients, who have an increased baseline risk of osteosarcoma, are not potential patients for PTH(1-34) formulations. Moreover, PTH(1-34) should not be given to patients with Paget's disease of bone or those who have elevated levels of alkaline phosphatase (above the upper limit of normal for the laboratory, which can be an indication of bone disease). Paget's disease of the bone is a chronic bone disorder in which bones become enlarged and deformed. Bone may become dense, but fragile, because of excessive breakdown and formation of bone. The disease affects both genders, is rarely found in people under age 40, and occurs in up to 3 percent of the US population. The exact cause of Paget's disease of the bone is not known, but it is suggested to be due to a slow viral infection of bone and may include a heredity factory. See http://www.methodisthealth.com/bone/pagets.htm.

In addition, pediatric patients or young adults with open epiphyses, i.e., the growth plates at the ends of the long bones, are also not good candidates for use of PTH(1-34), nor are patients with a prior history of radiation therapy involving the skeleton. Also excluded from treatment are subjects with bone metastases or a history of skeletal malignancies and subjects with metabolic bone diseases other than osteoporosis. Finally, hypercalcemic patients should not be treated with PTH(1-34). See e.g., http://www.rxlist.com/cgi/generic3/forteo pi.htm, and http://www.rphlink.com/forteo.html, as well as the labeling for Forteo®.

Thus, this invention is also directed to the discovery that certain dosages of PTH(1-84), potentially may be used to treat patient populations at risk of developing osteosarcoma or bone lesions, including: (1) subjects with Paget's disease of bone; (2) subjects with elevated levels of alkaline phosphatase; (3) pediatric subjects or young adults with open epiphyses; (4) subjects having a prior history of radiation therapy involving the skeleton; (5) subjects with bone metastases or a history of skeletal malignancies; and (6) subjects with metabolic bone diseases other than osteoporosis. Yet another embodiment of the invention is directed to a method for reducing the risk of osteosarcoma induced by an N-terminal PTH fragment in a subject, comprising administering to the subject a pharmaceutical composition comprising intact parathyroid hormone, i.e., PTH(1-84).

The invention also encompasses a method for reducing the risk of osteosarcoma induced by an N-terminal PTH fragment in a subject, comprising administering to the subject a pharmaceutical composition comprising a C-terminal fragment of parathyroid hormone.

Finally, the invention encompasses using safe dosages of full-length PTH(1-84), which do not result in an increased risk of osteosarcoma or bone lesions, as a preventative treatment for subjects having a gene which increases their likelihood of developing osteoporosis. As recently reported by DeCode Genetics (Iceland), subjects with a bad version of the gene BMP-2 were three times more likely to develop osteoporosis. Approximately 10% percent of the population is believed the have the bad versions of the BMP-2 gene. Because everyone's bones get thinner as they age, it is difficult to know in advance which patients will develop full-fledged bone disease. The bad version of the gene appears to boost osteoporosis risk by limiting the production of the BMP-2 protein, a key molecular stimulator of bone growth. This, in turn, limits a person's peak bone mass in adulthood, making osteoporosis a greater threat when bone density starts to decline late in life. See, for instance, information found in the following cite, http://www.forbes.com/2003/11/03/cz_rl₁₃1103decode.html.

### The PTH(1-84) Protein

As used herein, an “intact” or “full-length” parathyroid hormone is defined as a functional protein that comprises residues 1-84 of the mature human parathyroid hormone protein. The amino acid sequence of the PTH that may be used in the present invention is described in Kimura et al, Biochem Biophys Res Comm, 114 (2):493 (1983). Such a sequence is depicted in SEQ ID NO. 1.

SEQ ID NO. 1:
svseiqlmhnlgkhlnsmervewlrkklqdvhnfvalgapla
prdagsqrprkkednvlveshekslgeadkadvnvltkaksq

### PTH Variants

As an alternative to the full length human form of PTH, the preparation may incorporate those homologues or variants of human PTH that have human PTH activity, as determined in the ovarectomized rat model of osteoporosis reported by Kimmel et al, Endocrinology, 32(4):1577 (1993). According to the present invention, however, a variant or modified PTH protein should have a C-terminal end that is substantially similar to the C-terminus of the amino acid depicted in SEQ ID NO. 1. That is, any PTH variant, as well as modified PTH or PTH-fusion proteins, of the present invention should function similarly to native PTH(1-84) and have a similar anabolic effect and similar, if not the same, safety profile of PTH(1-84); namely, that there is no, or a minimal, increased risk in osteosarcoma or bone lesions, in individuals treated with the PTH(1-84) variant. One type of “C-terminal end” of full-length PTH could be a PTH fragment that contains residues 7-84 of the full-length protein.

Accordingly, the present invention encompasses the use of a PTH protein that has a sequence that is similar to, but not identical to, the amino acid sequence depicted in SEQ ID NO. 1. Thus, the present invention contemplates the use of a PTH protein that is functional but which has a sequence identity of 99%, 98%, 97%, 96%, 95%, 94%, 93%, 92%, 91%, 90%, 89%, 88%, 87%, 86%, 85%, 84%, 83%, 82%, 81%, or 80% compared to the sequence of SEQ ID NO. 1.

A PTH protein of the present invention may be modified to contain conservative variations or may be modified so as to change non-critical residues or residues in non-critical regions. Such modifications are described in detail in the art. See e.g., U.S. Pat. No. 6,331,427 to Robison. Amino acids that are not critical for function can be identified by methods known in the art, such as site-directed mutagenesis, crystallization, nuclear magnetic resonance, photoaffinity labeling, or alanine-scanning mutagenesis (Cunningham et al., Science, 244:1081-1085 (1989); Smith et al., J. Mol. Biol., 224:899-904 (1992); de Vos et al., Science, 255:306-312 (1992)). Modified proteins can be tested for biological activity via methods such as protease binding to substrate, cleavage, in vitro activity, or in vivo activity.

Specifically, a PTH variant may incorporate from 1 to 5 amino acid substitutions that improve PTH stability and half-life, such as the replacement of methionine residues at positions 8 and/or 18 with leucine, or with a different hydrophobic amino acid that improves PTH stability against oxidation, and the replacement of amino acids in the 25-27 region with trypsin-insensitive amino acids such as histidine, or with a different amino acid that improves PTH stability against protease. These forms of PTH are embraced by the term “parathyroid hormone” or “PTH(1-84)”as used generically herein.

Thus, a “variant” PTH polypeptide of the invention can differ in amino acid sequence from the sequence represented in SEQ ID NO. 1 by one or more substitutions, deletions, insertions, inversions, truncations, or a combination thereof. Any one of which can be made to contain amino acid substitutions that substitute a given amino acid with another amino acid of similar characteristics. Conservative substitutions include, among the aliphatic amino acids interchange of alanine, valine, leucine, and isoleucine; interchange of the hydroxyl residues serine and threonine, exchange of the acidic residues aspartate and glutamate, substitution between the amide residues asparagine and glutamine, exchange of the basic residues lysine and arginine, and replacements among the aromatic residues phenylalanine and tyrosine. See Bowie et al., Science, 247:1306-1310 (1990).

As noted above, a “variant,” according to the invention retains appropriate PTH biological activity, comprises the C-terminal end of PTH (or a substantial portion of the C-terminal end which produces the desired safety profile), and presents the safety profile described herein.

### PTH Fusion Proteins

A functional PTH polypeptide having the full-length sequence of SEQ ID NO. 1, or a functional variant thereof, can also be joined to another polypeptide with which it is not normally associated. Thus, a PTH protein can be operatively linked, at either its N-terminus or C-terminus, to a heterologous protein having an amino acid sequence not substantially homologous to the PTH. “Operatively linked” indicates that the PTH protein and the heterologous protein are both in-frame.

A fusion protein used in accordance with the invention should not affect the activity or safety of the full-length PTH, or a functional variant thereof. For example, the fusion protein may be a Glutathione S-transferase (GST)-fusion protein in which PTH(1-84) is fused to the C-terminus of the GST sequence or an influenza HA marker. Other types of fusion proteins include, but are not limited to, enzymatic fusion proteins, for example beta-galactosidase fusions, yeast two-hybrid GAL fusions, poly-His fusions, and Ig fusions. Such fusion proteins, particularly poly-His fusions, can facilitate the purification of recombinantly-produced PTH for use in the invention. In certain host cells, expression and/or secretion of a protein can be increased by using a heterologous signal sequence fused to a protease that transports the PTH protein to an extracellular matrix or localizes the PTH protein in the cell membrane.

PTH modifications

PTH variants also encompass derivatives or analogs in which: (i) an amino acid is substituted with an amino acid residue that is not one encoded by the genetic code, (ii) the mature polypeptide is fused with another compound, such as a compound that increases the half-life of the polypeptide (for example, polyethylene glycol), or (iii) the additional amino acids are fused to the mature polypeptide, such as a leader or secretory sequence or a sequence for purification of the mature polypeptide or a pro-protein sequence.

Typical modifications include, but are not limited to, acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphatidylinositol, cross-linking, cyclization, disulfide bond formation, demethylation, formation of covalent crosslinks, formation of cystine, formation of pyroglutamate, formylation, gamma carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination.

Particularly common peptide modifications that can be applied to PTH include glycosylation, lipid attachment, sulfation, gamma-carboxylation of glutamic acid residues, hydroxylation, and ADP-ribosylation. See T. E. Creighton, Proteins—Structure and Molecular Properties, 2nd Ed. (W. H. Freeman and Company, New York (1993)); Wold, F., Posttranslational Covalent Modification of Proteins, B. C. Johnson, Ed. (Academic Press, New York 1-12 (1983)); Seifter et al., Meth. Enzymol., 182: 626-646 (1990); and Rattan et al., Ann. N.Y. Acad. Sci., 663:48-62 (1992).

Modifications can be made anywhere in a PTH polypeptide, including the peptide backbone, the amino acid side-chains, and the amino or carboxyl termini. Blockage of the amino or carboxyl group in a polypeptide, or both, by a covalent modification, is common in naturally-occurring and synthetic polypeptides. However, any modification should retain appropriate PTH biological activity, comprise the C-terminal end of PTH (or a substantial portion of the C-terminal end which produces the desired safety profile) and present the safety profile described herein.

### Expression of PTH Proteins

PTH may be obtained through peptide synthesis or from genetically engineered systems, including yeast and bacterial hosts. Synthetic human PTH is commercially available (Bachem Inc., Switzerland). Recombinant human parathyroid hormone production is disclosed in the prior art, for example, EP 0383751, U.S. Pat. No. 5,223,407, and U.S. Pat. No. 5,629,205.

### PTH Formulations

The PTH(1-84) protein of the present invention may be formulated and stored as a lyophilized powder or a liquid, and may include a tonicity modifier, mannitol, and preservative, in a buffer of pH 4 to 6. Parathyroid hormone formulations are described in detail in U.S. Pat. No. 5,496,801.

Briefly, according to one aspect of the present invention, the PTH(1-84) preparation is in the form of a freeze dried composition, comprising a medically useful amount of full length parathyroid hormone (1-84) or a variant thereof, an excipient that will co-lyophilize with the parathyroid hormone to form an amorphous cake, and a non-volatile buffering agent in an amount sufficient to adjust the pH of the preparation to a physiologically acceptable pH. Preferably, the hormone in the preparation is human parathyroid hormone, the excipient is mannitol, and the buffering agent is a citrate source. In addition, preferably a tonicity modifier, such as NaCl is included.

The PTH(1-84) or a variant thereof may be formulated with the buffering agent, excipient, and tonicity modifier, and then subjected to a freeze-drying process that yields a product incorporating less than 2% water by weight. The freeze-dried PTH(1-84) protein or variant thereof can then be reconstituted in sterile water. The formulated PTH(1-84) can then be appropriately packaged for patient use with instructions for administration.

The excipient incorporated in the preparation serves as a cryoprotectant during the freeze-drying process and also as a bulking agent to facilitate dosage formulation. In having selected the excipient on this basis, the cake resulting from the freeze-drying process is of the homogeneous quality desired for rapid reconstitution. Polyol-type excipients are preferred. An evaluation of caking properties of polyol-type excipients has revealed that mannitol is a particularly preferred excipient, not only for its ability to yield a quality cake, but also because mannitol itself confers some stability to PTH(1-84) in solution.

The buffering agents incorporated in the present preparations are selected from those capable of buffering the preparation to a pH within a physiologically acceptable range. A pH that is physiologically acceptable is that which causes either no, or minimal, patient discomfort when the formulation is administered, and can thus vary depending on the mode of administration. For preparations that will be diluted prior to administration, such as by dissolution in a stock infusion solution, the pH of the preparation per se can vary widely, e.g., from about pH 3 to about pH 9. Where the preparation is to be administered directly after reconstitution, the PTH preparation is buffered desirably to within the pH range from 3.5 to 7.5. Suitable buffers are accordingly those pharmaceutically acceptable agents that can buffer the pH of the preparation to within the target pH range, and include phosphate-based buffers and, preferably, citrate-based buffers such as sodium citrate/citric acid.

In a particular embodiment of the invention PTH(1-84) is formulated with mannitol, citrate and sodium chloride. In particular, PTH(1-84) may be formulated as a multi-dose composition. In a particular multi-dose composition 100 ug of PTH(1-84) is formulated in a 14 day multi-dose composition comprising mannitol, sodium chloride and citrate buffer. See U.S. Pat. No. 5,496,801 and PCT/CA99/00376, incorporated herein by reference.

### Supplemental Substances

The PTH(1-84) preparation can be co-formulated or co-administered together with or separately with other substances that aid treatment of the recipient. That is, the present invention encompasses the co-formulation or co-administration of a substance that enhances the effect of PTH(1-84) and/or counters any side-effects of PTH(1-84) administration. For instance, such a supplemental substance may be an antireabsorbative substance that helps control further loss of bone. See, for example, U.S. Pat. No. 6,284,730.

By the same token, the co-administration, either simultaneously or sequentially, of a supplemental substance with PTH(1-84) could facilitate the administration of a larger dose of PTH(1-84) than when the substance is absent or not incorporated in the treatment regime. This is because the supplemental substance could act to relieve or counter any side-effects that a larger dose may have upon physiological, biochemical, or molecular aspects of bone formation or bone modeling in the treated individual. For example, a PTH(1-84) pharmaceutical composition together with a substance that helps control high levels of calcium may be desirable for administration to an individual. In particular, such a combination may be administered simultaneously or sequentially to an individual that might be at risk of hypercalcemia or whose blood calcium levels increase after treatment with PTH(1-84).

Thus, suitable supplemental substances include, but are not limited to, vitamin D, dietary calcium, bisphosphonates, alendronate, estrogen, selective estrogen receptor modulators, raloxifene, tamoxitene, droloxifene, calcitonin, R1, BIS, GM-CSF, hGRF(144)-NH-2 or analogs thereof, benzo-fused lactam, 5-AR-I inhibitors, benzoquinolin-3-ones, VEGF, GLP-2, or a combination thereof. For instance, it is known that alendronate functions synergistically with vitamin D and calcium. Accordingly, one PTH(1-84) formulation of the present invention is a mixture of PTH(1-84), alendronate, and other supplemental substance(s), such as vitamin D and/or calcium. Such substances may be incorporated into a PTH(1-84) formulation or administered simultaneously, sequentially, or independently with various dosages of PTH(1-84).

### Dosage Regimes for PTH(1-84)

A dose of an intact, full-length parathyroid hormone may be administered to a male or female subject of any age.

The intensity of a concomitant response to PTH, or the likelihood that a biological bone-related response will be generated in an individual treated with PTH, is tied to the level of exposure (“AUC”) to PTH. The amount of PTH present in the circulation and the length of time the PTH is present in the circulation is an indicator of such “exposure.” After administration, usually via subcutaneous injection, full-length PTH is active and circulates throughout the blood system, whereupon it eventually is processed according to the body's normal biological mechanisms. By standardizing the level of PTH to which an individual is exposed, it is possible to develop a treatment regime based upon level of exposure, rather than definitive dosage amounts. Typically, a blood sample is taken at various time points after administration, from which the concentration of PTH is measured. That measurement reflects the “exposure” value of PTH at that time point and that administered dosage.

An AUC ratio between rats and humans helps standardize the effects of PTH between treated non-human animal models and human clinical trials. For example, a 100 μg dose of PTH(1-84) in a human is equivalent to an exposure, i.e., AUC, of approximately 1 ng/hr/ml. The AUC exposure value of the same or different dose of PTH to a rat can be used to create a ratio between the human 1 ng/hr/ml AUC value and the rat's AUC value for that particular dose. Accordingly, an AUCᵣₐₜ:AUCₕᵤₘₐₙ ratio of “2,” for instance, means that that rat was exposed to twice as much PTH than the human subject.

It was found that a 10 μg/kg/day dosage of PTH(1-84) in rats compared to a 100 μg/day dosage of PTH(1-84) in humans results in a AUCᵣₐₜ:AUCₕᵤₘₐₙ ratio of approximately 3.70. See the Examples which follow below.

An AUCᵣₐₜ:AUCₕᵤₘₐₙ exposure ratio can also be used to determine safe dosages of PTH(1-84) for humans based on safe dosages of PTH(1-84) for rats. For example, it was determined in the studies described herein, that the 10 82 g/kg/day dosage of PTH(1-84) did not result in any increase in incidence of osteosarcoma in rats beyond the normal background incidence of osteosarcoma recorded for rats. Hence, even though the rat was exposed to approximately 3.70 times more PTH(1-84) than a human who receives a 100 μg dose, no increase in bone cancer beyond background was detected. Thus, it can be predicted that a 370 μg dose of PTH(1-84) to a human, i.e., equivalent to an exposure of 3.70, also would not cause an increased chance of developing bone cancer beyond background.

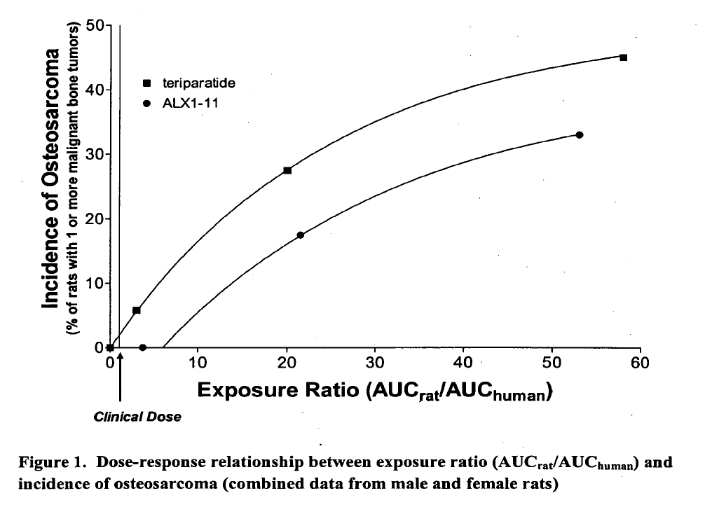

As the average human weight is about 70 kg, then a 100 μg/day dose is equivalent to about 1.4 μg/kg/day. According to the results described herein, a rat can tolerate up to at least about six-times the exposure to PTH than that created by a 100 μg dose in a human, before the incidence of osteosarcoma begins to increase beyond background (see FIG. 1). Therefore, an upper safety limit for PTH(1-84) dosage for a human, which does not result in an increased risk for osteosarcoma or bone lesions, is 8.4 μg/kg/day, or 600 μg/person/day.

Accordingly, the present invention contemplates dosages of PTH(1-84) in humans of about 0.1, 0.2, 0.3, 0.4, 0.5, 0.6, 0.7, 0.8. 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3 and 8.4 μg/kg/day. Preferably, the dose of PTH(1-84) administered to a human is about 0.5, 0.6, 0.7, 0.8, 0.9, 1.0, 1.1, 1.2, 1.3, 1.4, 1.5, 1.6, 1.7, 1.8, 1.9, and 2.0 μg/kg/day. More preferably, the dosage of PTH(1-84) administered to a human is about 0.7, 0.8, 0.9 1.0, 1.1, 1.2, 1.3, 1.4, 1.5, 1.6, and 1.7 μg/kg/day.

At higher-end dosages, i.e., greater than 3.0 μg/kg/day, or greater then 200 μg/day, the PTH(1-84) may be combined, physically or regimentally, with one or more of the supplemental substances described herein. This means the supplemental substance may be formulated into the same composition as the PTH(1-84), i.e., “physically,” or administered to the individual as separately from the composition comprising the PTH(1-84) protein, either simultaneously or sequentially, i.e., “regimentally.” In an embodiment of the invention, if hypercalcemia is an issue in those individuals receiving higher doses of PTH(1-84), then PTH(1-84) may be combined physically or regimentally with one or more supplemental substances to actively decrease calcium levels. Similarly, the preferred actual dosage level of an intact, full-length parathyroid hormone to a human is about 1, about 2, about 3, about 4, about 5, about 6, about 7, about 8, about 9, about 10, about 11, about 12, about 13, about 14, about 15, about 16, about 17, about 18, about 19, about 20, about 25, about 30, about 35, about 40, about 45, about 50, about 55, about 60, about 65, about 70, about 75, about 80, about 85, about 90, about 95, about 100, about 110, about 120, about 130, about 140, about 150, about 160, about 170, about 180, about 190, about 200, about 210, about 220, about 230, about 240, about 250, about 260, about 270, about 280, about 290, about 300, about 350, about 400, about 450, about 500, about 550 or about 600, μg/person/day, or any integer in between. An upper safety limit for a PTH(1-84) dosage for a human with no increased risk in osteosarcoma is about 600 μg/person/day, or about 400 μg/person/day, or about 370 μg/person/day, or about 200 μg/person/day. However, this upper dosage may be reduced in an effort to minimize hypercalcemia effects, which have generally been seen at dosages above 200 μg/person/day. A preferable dosage for administration of PTH(1-84) to a human is any dosage from about 25 μg/person/day to about 200 μg/person/day. An additional preferable dosage for administration of PTH(1-84) to a human is 25-150 μg/person/day, more preferably 50-100 μg/person/day, and most preferably is 100 μg/person/day.

Also, the dosage concentration of an intact, full-length parathyroid hormone administered to a human may be about 0.30, about 0.31, about 0.32, about 0.33, about 0.34, about 0.35, about 0.36, about 0.37, about 0.38, about 0.39, about 0.40, about 0.41, about 0.42, about 0.43, about 0.44, about 0.45, about 0.46, about 0.47, about 0.48, about 0.49, about 0.50, 0.51, about 0.52, about 0.53, about 0.54, about 0.55, about 0.56, about 0.57, about 0.58, about 0.59, about 0.60, about 0.61, about 0.62, about 0.63, about 0.64, about 0.65, about 0.66, about 0.67, about 0.68, about 0.69, about 0.70, about 0.71, about 0.72, about 0.73, about 0.74, about 0.75, about 0.76, about 0.77, about 0.78, about 0.79, about 0.80, about 0.81, about 0.82, about 0.83, about 0.84, about 0.85, about 0.86, about 0.87, about 0.88, about 0.89, about 0.90, about 0.91, about 0.92, about 0.93, about 0.94, about 0.95, about 0.96, about 0.97, about 0.98, about 0.99, about 1.0, about 1.1, about 1.2, about 1.3, about 1.4, about 1.5, about 1.6, about 1.7, about 1.8, about 1. 9 or about 2.0 mg/ml. Preferably the dosage concentration is about 1.0, about 1.1, about 1.2, about 1.3, about 1.4, about 1.5, about 1.6, about 1.7 or about 1.8 mg/ml. Most preferably the dosage concentration is about 1.4 mg/ml.

The dosage regime for administering an intact, full-length parathyroid hormone to a human may entails administering the dose to the subject once a day for about 1, about 2, about 3, about 4, about 5, about 6, about 7, about 8, about 9, about 10, about 11, about 12, about 13, about 14, about 15, about 16, about 17, about 18, about 19, about 20, about 25, about 30, about 35, about 40, about 45, about 50, about 55, about 60, about 65, about 70, about 75, about 80, about 85, about 90, about 95, about 100, about 110, about 114, about 120, about 130, about 140, or about 150 weeks. Also, a dosage regime in months for administering an intact, full-length parathyroid hormone to a human is preferably about 3, about 6, about 9, about 12, about 15, about 18, about 21, about 24 months, about 27 months, about 30 months, about 33 months or about 36 months. These dosage regimes may entail administering the dose to the subject once, twice, or multiple times a day. Preferably the dose is administered once a day.

Alternatively, the dosage regime for administering an intact, full-length parathyroid hormone to a human may entail administering the composition for an indefinite time period on a continuous or intermittent schedule to counteract bone loss.

The dosage regime may also be staggered so that there exists 1, 2, 3, 4, 5, 6, 7, 8, 9, 10, 11, 12, 13, 14, or more days between single or multiple administrations of an intact PTH dosage.

In the experiments described below, groups of rats were each given PTH(1-84) dosages of either 10 μg/kg/day, 50 μg/kg/day, or 150 μg/kg/day, which represent the molar equivalent dosages of 5 μg/kg/day, 30 μg/kg/day, and 75 μg/kg/day of teriparatide, i.e., PTH(1-34) (Forteo™), respectively.

Strikingly, the data presented herein, which represent the results from a 2-year rat carcinogenicity study conducted with PTH(1-84), demonstrate that certain dosages of full-length PTH result in no increased incidence of osteosarcoma beyond background. In contrast, the Forteo™ teriparatide product, PTH(1-34), was found to increase the incidence of bone cancer in rats.

Substantial dose-related increases in bone area, bone mineral content and bone mineral density were observed in rats given PTH(1-84). Moreover, histopathologic evidence indicated that all doses of PTH(1-84) induced extensive new bone growth at multiple skeletal sites. These rats experienced a nearly 4-fold greater exposure at the low dose (10 μg/kg/day), a 22-fold grater exposure at the mid dose (50 μg/kg/day) and a 53-fold greater exposure at the high dose (150 μg/kg/day) when compared with the exposure in humans following administration of the 100 μg clinical dose of PTH(1-84) (see Table 3).

Importantly, the present data indicate that the rats in this study received exposure ratios of PTH(1-84) that were very comparable to those reported previously in the PTH(1-34) carcinogenicity study (Vahle et al., supra). For example, in the PTH(1-34) study, the low dose (5 μg/kg/day) resulted in a systemic PTH(1-34) exposure approximately 3-fold greater than that which occurred in humans given the prescribed 20 μg dose. Moreover, the lowest doses in both studies (5 μg/kg/day dose of PTH(1-34) and 10 μg/kg/day of PTH(1-84)) resulted in substantial new bone growth as a pharmacological response to drug treatment.

Since all doses of PTH(1-34) tested in the 24-month rat study resulted in a substantial increase in the incidence of osteosarcoma, no dosage or exposure “safety margin” could be established for human administration. Therefore, it had to be assumed that any exposure to PTH(1-34) in humans could increase the incidence of osteosarcoma. FIG. 1 illustrates the observed data between the present study with PTH(1-84) and the results for PTH(1-34).

The following examples are given to illustrate the present invention. It should be understood, however, that the invention is not to be limited to the specific conditions or details described in these examples. Throughout the specification, any and all references to a publicly available document, including a U.S. patent, are specifically incorporated by reference.

### EXAMPLE 1

The purpose of this example was to investigate the carcinogenic potential of PTH(1-84). Groups of male and female rats were subjected to different doses of PTH(1-84) and studied for up to 104 weeks or two (2) years. In general, the dosages of PTH(1-84) used in this experiment are at least 3-fold greater than a dose of 100 μg/day that would be administered to a human.

### A. The Test System

Rats of the species Rattus norvegicus, strain Fischer 344 (F344/NHsd), obtained from Harlan Sprague Dawley (Indianapolis, Ind.) were used in the experiment. The animals were approximately 9 to 11 weeks at the onset of treatment. At the onset of treatment the male rats were approximately 120-240 g, and the female rats were approximately 100-220 g. An exemplary 10 males and 10 females were subjected to a health screen, as described in more detail below.

In the study design, six (6) groups were used, with a total of sixty (60) rats/sex/group assigned to the main study, 24 rats/sex for Groups 2 and 5, and 8 rats/sex for Groups 3 and 4 were assigned to the toxicokinetic study.

Following arrival, each animal was given a general physical examination by a member of the veterinary staff to assess health status.

### B. Dietary Materials

All animals had free access to a standard certified pelleted commercial laboratory diet (PMI Certified Rodent Chow 5002: PMI Feeds Inc.) except during designated procedures. Water bottles and/or powdered diet (PMI Rodent Laboratory Meal 5002) were provided, if needed. Municipal tap water which had been softened, purified by reverse osmosis, and exposed to ultraviolet light was freely available to the animals.

The maximum allowable concentrations of contaminants in the diet (e.g., heavy metals, aflatoxin; organophosphate, chlorinated hydrocarbons, PCBs) were controlled and routinely analyzed. It is considered that there were no known contaminants in the dietary materials that could interfere with the objectives of the study

### C. Assignment to Groups

A minimum acclimation period of 10 days was allowed between animal receipt and the start of treatment to accustom the animals to the laboratory environment.

Prior to the start of treatment, 10 male and 10 female animals were selected from the total population using computer based random numbers for the provision of blood samples and gross pathology examination for health-screen purposes.

In addition, prior to treatment initiation, the animals were weighed and assigned to treatment groups using a randomization procedure. Randomization was by stratification using body weight as the parameter. Males and females were randomized separately. Animals in poor health or at the extremes of the body weight range were not assigned to groups.

Animals were randomized into the following groups.

TABLE 1

| | | | Dose Concen- | Main Study | | Toxicokinetic Study | |
|---|---|---|---|---|---|---|---|
| Group No. | | Dose Level | tration | Animal Numbers | | Animal Numbers | |
| Identification | | (μg/kg/day) | (mg/mL) | Males | Females | Males | Females |
| 1 | Vehicle | 0 | 0 | 1001-1060 | 1501-1560 | — | — |
| | Control | | | | | | |
| 2 | Vehicle | 0 | 0 | 2001-2060 | 2501-2560 | 2061-2084 | 2561-2584 |
| | Control | | | | | | |
| 3 | PTH(1-84)- | 10 | 0.05 | 3001-3060 | 3501-3560 | 3061-3068 | 3561-3568 |
| | Low Dose | | | | | | |
| 4 | PTH(1-84)- | 50 | 0.25 | 4001-4060 | 4501-4560 | 4061-4068 | 4561-4568 |
| | Mid Dose | | | | | | |
| 5 | PTH(1-84)- | 150 | 0.75 | 5001-5060 | 5501-5560 | 5061-5084 | 5561-5584 |
| | High Dose | | | | | | |
| 6 | PTH(1-84)- | 150 | 0.75 | 6001-6060 | 6501-6560 | — | — |
| | High Dose* | | | | | | |
| 7 | Health | — | — | 7001-7010 | 7501-7510 | — | — |
| | Screen | | | | | | |

*Animals commenced dosing on study Week 24 (at approximately 8 months of age) and continued with treatment until the completion of dosing for the remaining groups (at least up to study Week 104). From study Weeks 1 to 23, Group 6 animals remained untreated but were held in the dosing position every day at the time of dosing.

Prior to initiation of dosing, any assigned animals considered unsuitable for use in the study were replaced by spare animals obtained from the same shipment and maintained under the same environmental conditions.

After initiation of dosing, assigned animals that died or were euthanized were replaced with spare animals during Days 1 to 14. All animals remaining unassigned to groups after Day 14 were released from the study and their disposition documented.

Any animal found dead or euthanized prior to the start of treatment or replaced following the start of treatment was subject to necropsy and tissue retention.

### D. Vehicle/Control and Test Articles

The Vehicle/Control Article was citric acid in D-Manitol (USP), which was stored under refrigerated conditions.

The test article was labeled “ALX1-11” (PTH(1-84)), and was stored under frozen conditions (approximately −20° C.), and exposed to room temperature on day of dosing. Samples of test article (one vial) were retained both before and after the treatment period. As used herein, the terms “ALX1-11” and “PTH(1-84)” are synonymous.

### E. Preparation of Dose Formulations

The dose formulations were prepared daily and the vehicle was prepared weekly. Prior to the study start, the PTH(1-84) concentration in each dose solution following the formulation procedure to be used during the study was verified. One sample (approximately 0.5 mL) was collected from the container prior to dosing of each dose formulation prepared for Weeks 1, 13, 26, 39, 52, 65, 78, 91, and 104 for concentration verification. The remainder of the vial after dosing was frozen at approximately −20° C. and the 0.5 mL sample retained frozen at approximately −20° C.

### F. Administration of Test Article

The test/control articles were administered by subcutaneous injection at a dose volume of 200 μg/kg into the dorsal region daily for up to 104 weeks for the main study animals and up to 52 weeks for the toxicokinetic animals. Injection sites were rotated daily between 7 areas on the back using a predetermined schedule. The actual dose administered was based on the most recently measured body weight of each animal. The first day of dosing was designated as Day 1.

Animals in Group 6 commenced dosing on study Week 24 (at approximately 8 months of age) and continued with treatment until the completion of dosing for the remaining groups (at least up to study Week 104). From study Weeks 1 to 23, Group 6 animals remained untreated but were held in the dosing position every day at the time of dosing.

### G. Observations

Records of activities relating to the day-to-day running and maintenance of the study within the animal room, as well as the activities relating to the observations and examinations outlined in this protocol, were recorded. During the study, additional evaluations to those described below and/or scheduled, and considered necessary were conducted and duly documented.

1. Clinical Examination

All animals were examined twice daily for mortality and signs of ill health or reaction to treatment. A complete detailed examination was performed weekly commencing one week prior to treatment initiation. More frequent observations were undertaken if considered appropriate. In addition, from Week 26 onwards, all main study animals were examined for the presence of palpable masses weekly during the detailed examination. The site, size, and appearance of these masses was recorded when first detected and, following this initial description, the presence or disappearance of these masses was monitored. Any mass borne by an animal was given a numerical designation (e.g., M₁, M₂, etc.) according to order of appearance in that animal. Death and observed clinical signs was individually recorded.

Moribund animals were euthanized for humane reasons and animals were subjected to detailed external and internal gross examinations. 2. Body Weight

Individual body weights were measured weekly commencing on the day of randomization and extending through the first 13 weeks of treatment and monthly thereafter.

3. Food Consumption

Individual food consumption was measured during the week prior to treatment initiation and weekly throughout the first 13 weeks of treatment, thereafter the measurements were performed once monthly.

4. Laboratory Investigations

Red blood cell count and total and differential white blood cell counts (including blood cell morphology) were performed on health screen animals (10 per sex), all surviving toxicokinetic animals at 12 months, and main study animals at 24 months. Blood samples were collected for biochemistry evaluations at Month 24 on main study animals only at terminal sacrifice. In addition, blood samples for hematology and biochemistry were collected if possible from main study animals euthanized early. If the analyses could not be performed on the same day as the unscheduled euthanasia, then blood samples were refrigerated until assessment was possible.

Blood samples were collected from the abdominal aorta following isoflurane anesthesia at necropsy.

For all main and toxicokinetic animals euthanized, 3 femoral bone marrow smears were prepared, one of which was stained with May-Grünwald-Giemsa. The smears were retained and evaluated only if needed for diagnostic purposes by the study pathologist.

5. Clinical Biochemistry

The biochemical parameters examined included: (1) AUC ratio (calculated); (2) alanine aminotransferase albumin; (3) alkaline phosphatase; (4) aspartate aminotransferase blood urea nitrogen; (5) calcium; (6) chloride; (7) cholesterol; (8) creatinine; (9) globulin (calculated) glucose; (10) inorganic phosphorus potassium; (11) sodium; (12) total bilirubin; (13) total protein; and (15) triglycerides.

6. Biochemical Markers of Bone Turnover

Blood samples were collected from all surviving main study animals at month 24 in the morning prior to final euthanasia. Blood samples were collected from the jugular vein without anesthesia and following an overnight deprivation of food, and placed on wet ice following collection and prior to processing. In addition, blood samples were collected if possible from main study animals euthanized early.

Any serum samples remaining following completion of all parameters were stored frozen at approximately −20° C. for possible future analyses. Remaining samples were discarded following completion of the study.

The parameters examined included: (1) the serum bone formation markers osteocalcin and total and bone specific alkaline phosphatase; and (2) the serum bone resorption marker C-Telopeptide.

7. Radiographs

Radiographs of the whole skeleton (dorsa/ventral and lateral) were performed under isoflurane anesthesia during the two weeks prior to necropsy on all surviving main study (Month 24) and toxicokinetic animals euthanized Month 12.

8. Toxicokinetics

Blood samples (approximately 1 mL) were collected from toxicokinetic animals at the timepoints specified in the following table. Samples were collected from the jugular vein without anesthesia into tubes containing EDTA. On each occasion (Day 7, Months 1, 6, and 12), samples were collected from 3 (Groups 2 and 5) or 1 (Groups 3 and 4) animals as shown in the table below.

In addition, a blood sample was collected during the last week of the treatment period for all surviving main study animals for toxicokinetic evaluation. The dosing and blood collection times were recorded. The samples were centrifuged at 2700 rpm at approximately 2-8° C. for 10 minutes, the collected plasma was placed on dry ice, and then stored frozen at −80° C.

Once the final toxicokinetic blood samples were collected (Month 12), the toxicokinetic animals were euthanized following radiographic evaluation and blood collection for hematology evaluation.

TABLE 2

| | | Timepoints (minutes post dosing) | | | |
|---|---|---|---|---|---|
| Groups 2 and 5 | Groups 3 and 4 | Day | Month | Month | Month |
| Rat Numbers* | Rat Numbers* | 7 | 1 | 6 | 12 |
| 61-63 | 61 | 0 | 60 | 150 | 30 |
| 64-66 | 62 | 5 | 90 | 120 | 15 |
| 67-69 | 63 | 15 | 120 | 90 | 5 |
| 70-72 | 64 | 30 | 150 | 60 | 0 |
| 73-75 | 65 | 60 | 0 | 30 | 150 |
| 76-78 | 66 | 90 | 5 | 15 | 120 |
| 79-81 | 67 | 120 | 15 | 5 | 99 |
| 82-84 | 68 | ISO | 30 | 0 | 60 |

*Last 2 digits of the animal number

Non-compartmental toxicokinetic analysis were performed on the total PTH(1-84) (including background) plasma concentration data. Toxicokinetic analysis included assessment of the tₘₐₓ, Cₘₐₓ, and AUC. The tₘₐₓ and Cₘₐₓ are observed values. The AUC parameter was calculated by the trapezoidal rule method (Gibaldi, M. and Perrier, D., Pharmacokinetics, Second Edition In: Drugs and the Pharmaceutical Sciences, vol. 15, Ed. Swarbick, J. (Marcel Dekker Inc., NY, 1982) using the standard computer software program WinNonlin (Version 1.5A).

9. Tissue Preservation

On completion of the necropsy of each animal, tissues and organs were retained. If necessary, additional tissue samples were taken at the discretion of the attending pathologist to elucidate abnormal findings.

In addition, each clinically observed mass together with the nearest identifiable drainage lymph node was preserved and labeled according to the numerical designation on the animal's clinical observation sheet to facilitate future identification.

A gross examination of bones was conducted during the necropsy for signs of osteosarcoma. For all euthanized animals, 3 femoral bone marrow smears were prepared, one of which was stained with May-Grünwald-Giemsa. The smears were retained and evaluated only if needed for diagnostic purposes by the study pathologist.

10. Histopathology

All specified tissues from all animals were prepared for histopathological examination by embedding in paraffin wax, sectioning, and staining with hematoxylin and eosin and examined as follows;
(a) All animals from Groups 1, 2, 5, and 6 (controls and high-dose groups).
(b) All animals in Groups 3 and 4 (low-and intermediate-dose groups) which died or were euthanized prior to study termination.
(c) All gross findings in all groups.

Target organs identified in high-dose animals following examination were similarly examined in low- and intermediate-dose groups. All remaining tissues which were not examined were retained as wet tissues (no processing was performed). All suspected tumors were diagnosed and the incidences of benign and malignant tumors of different cell types in the various treatment groups were tabulated. Histopathological assessment of selected bones was conducted specifically to look for signs of osteosarcoma.

11. Bone Mineral Density Measurements (BMD)

Bone mineral density measurements (ex vivo) were performed at Month 24 on all main study animals euthanized at the end of the treatment period. Bone mineral density measurements were performed using an Hologic QDR-2000 plus densitometer.

The ex vivo DXA scans were used to measure the bone mineral density of the right proximal femur, the right central femur, the right distal femur and L1 to L4 vertebrae, DXA scans were not be performed on any animals found dead or euthanized for humane reasons during the study or at Month 12. The right femur and the vertebrae L1 to L4 were retained in neutral buffered 10% formalin overnight and then transferred to 70% alcohol and then DXA scanned.

### H. Results

1. Toxicokinetics

Systemic exposure measurements were taken on Day 7, and Months 1, 6 and 12. As is common in 2-year rodent studies, no exposure measures were taken after 12 months to avoid the potentially confounding effects of changes in disposition at an advanced age. As such, the 6- and 12-month data were averaged to obtain a reasonable estimate of exposure to PTH(1-84) over the study. This is consistent with the data used in the “Vahle” teriparatide study, where the data from 6, 12 and 18 months were averaged. Table 3 summarizes the exposure ratios, AUCᵣₐₜ AUCₕᵤₘₐₙ, following PTH(1-84) administration and compares them with the exposure ratios of teriparatide.

TABLE 3
PTH(1-84) exposure (AUC) ratios* at 6 and 12 month
as compared with the exposure ratio of PTH(1-34)

| PTH(1-84) | | | | | |
|---|---|---|---|---|---|
| | Exposure Ratio | | | PTH(1-34) | |
| Dose | Month | Month | | Dose | Exposure ratio |
| (μg/kg/day) | 6 | 12 | Mean | (μg/kg/day) | Mean** |
| 10 | 3.65 | 3.75 | 3.70 | 5 | 3 |
| 50 | 22.1 | 20.9 | 21.5 | 30 | 20 |
| 150 | 48.3 | 58.0 | 53 | 75 | 58 |

*Exposure ratios = AUCᵣₐₜ/AUCₕᵤₘₐₙ; where AUCₕᵤₘₐₙ for PTH(1-84) is estimated to be approximately 1 ng · hr/mL, based on Phase I clinical data.
**Average of Month 6, 12 and 18.

### Toxicology

Gross necropsy, body weight, blood chemistry, and other toxicologic data are not yet available; however, the high rate of mortality in the high-dose male group suggest that the maximum tolerated dose (MTD) was exceeded in this group and required termination of dosing during Week 94 and early sacrifice at the end of Week 101.

3. Pathology

Widespread, dose-related osteosclerosis, involving both the axial and appendicular skeleton, was noted during radiological examination of the animals receiving PTH(1-84). This finding was confirmed at necropsy, which revealed markedly increased cortical thickness with a concurrent reduction of the medullary space, particularly with the 50 and 150 μg/kg doses. Two rats in the Female Control Group 2 developed osteosarcoma over the 2-year study. Primary bone neoplasms and focal osteoblast hyperplasia were observed in both male and female rats receiving the high dose of PTH(1-84) and to a lesser extent in rats receiving the intermediate dose. Two male rats that received the low dose of PTH(1-84) developed malignancies. However, neither of the masses were considered to have a primary bone origin. Indeed, one of the malignancies in the low-dosed rat was observed in the lung, while the other mass was found in subcutaneous tissue in the cervical region. The latter mass was well-encapsulated and atypical of the osteosarcomas seen in bone or in other soft tissues. Tables 4 and 5 summarize the incidence of malignant and benign bone neoplasms by dose group.

TABLE 4
Incidence in male rats of bone neoplasms at all sites
in 2-year carcinogenicity study with ALX1-11

| | MALES | | | | | |
|---|---|---|---|---|---|---|
| | Group | | | | | |
| | 1 | 2 | 3 | 4 | 5 | 6 |
| | Dose (μg/kg/day) | | | | | |
| | Control | Control | Low | Mid | High | High |
| | (0) | (0) | (10) | (50) | (150)^{a} | (150)^{b} |
| No. Examined | 60 | 60 | 60 | 60 | 60 | 60 |
| No. of animals with: | | | | | | |
| Osteosarcoma | 0 | 0 | 2^{c} | 13 | 27 | 13 |
| Total malignant | 0 | 0 | 2^{c} | 14 | 27 | 13 |
| bone tumors^{d} | | | | | | |
| Benign bone | 0 | 0 | 0 | 3 | 6 | 3 |
| tumors^{e} | | | | | | |

^{a}Treatment was terminated during Week 94 and all surviving animals were sacrificed at the end of Study Week 101.
^{b}Animals in this group began dosing on Study Week 24 (at approximately 8 months of age) and continued for a minimum for 80 weeks.
^{c}Neither mass was observed to have a primary bone origin. One was observed in the lung while the other mass was found in subcutaneous tissue in the cervical region. The latter mass was well-encapsulated and atypical of the osteosarcomas seen in bone or in other soft tissues.
^{d}Skeletal and extraskeletal osteosarcoma, skeletal fibrosarcoma and/or osteoclastoma (soft tissue reads are complete for Groups 1, 2, and 3 only).
^{e}Osteoblastoma and/or osteoma.

TABLE 5
Incidence in female rats of bone neoplasms at all
sites in 2-year carcinogenicity study with ALX1-11

| | FEMALES | | | | | |
|---|---|---|---|---|---|---|
| | Group | | | | | |
| | 1 | 2 | 3 | 4 | 5 | 6 |
| | Dose (μg/kg/day) | | | | | |
| | Control | Control | Low | Mid | High | High |
| | (0) | (0) | (10) | (50) | (150) | (150)^{a} |
| No. Examined | 60 | 60 | 60 | 60 | 60 | 60 |
| No. of animals with: | | | | | | |
| Osteosarcoma | 2 | 0 | 0 | 5 | 13 | 19 |
| Total malignant | 2 | 0 | 0 | 7 | 13 | 20 |
| bone tumors^{b} | | | | | | |
| Benign bone | 0 | 0 | 0 | 4 | 10 | 1 |
| tumors^{c} | | | | | | |

^{a}Animals in this group began dosing on Week 24 (at approximately 8 months of age) and continued for a minimum for 80 weeks.
^{b}Skeletal and extraskeletal osteosarcoma, skeletal fibrosarcoma and/or osteoclastoma (soft tissue reads are complete for Group 3 only).
^{c}Osteoblastoma and/or osteoma.

4. Densitometry

Preliminary data from the DXA assessment indicated that bone area, BMC and BMD at the lumbar vertebra and femur were virtually identical in the two vehicle-treated groups. Treatment with PTH(1-84) for up to 2 years induced substantial dose-related increases in bone area, BMC and BMD in male and female rats at lumbar vertebrae and at each region of the femur. The increases in BMC relative to that in vehicle-dosed rats are summarized in Tables 6 and 7. Of note, the increase in BMC was similar in Groups 5 and 6 that received the 150 μg/day dose of PTH(1-84) starting at approximately 2 and 8 months of age, respectively.

TABLE 6
Preliminary results relating to increases in bone mineral content
in male rats in 2-year carcinogenicity study with ALX1-11

MALES

| Group | 3 | 4 | 5 | 6 |
|---|---|---|---|---|
| Dose | Low | Mid | High | High |
| (μg/kg/day) | (10) | (50) | (150)^{a} | (150)^{b} |
| N | 16 | 16 | 14 | 18 |
| BMC (% increase) | | | | |
| Lumbar vertebra-3 | 24 | 51 | 70 | 69 |
| Proximal femur | 16 | 58 | 85 | 85 |
| Central femur | 21 | 66 | 112 | 109 |
| Distal femur | 23 | 83 | 120 | 115 |

^{a}Treatment was terminated during Week 94 and all surviving animals were sacrificed at the end of Study Week 101.
^{b}Animals in this group began dosing on Week 24 (at approximately 8 months of age) and continued for a minimum for 80 weeks.

TABLE 7
Preliminary results relating to increases in bone mineral content
in female rats in 2-year carcinogenicity study with ALX1-11

FEMALES

| Group | 3 | 4 | 5 | 6 |
|---|---|---|---|---|
| Dose | Low | Mid | High | High |
| (μg/kg/day) | (10) | (50) | (150) | (150)^{a} |
| n | 27 | 21 | 24 | 29 |
| BMC (% increase) | | | | |
| Lumbar vertebra-3 | 26 | 52 | 76 | 73 |
| Proximal femur | 15 | 34 | 60 | 59 |
| Central femur | 18 | 46 | 79 | 77 |
| Distal femur | 20 | 49 | 77 | 75 |

^{a}Animals in this group began dosing on Week 24 (at approximately 8 months of age) and continued for a minimum for 80 weeks.

Increases in trabecular and cortical bone mass are expected consequences of long-term administration of ALX1-11, i.e., PTH(1-84), or N-terminal PTH analogs to rats. See, Kimmel et al., Endocrinology, 132, pp. 1577-1584, 1993 and Ejersted et al., J. Bone Miner. Res., 8, pp. 1097-1101, 1993. Indeed, in the present study, substantial dose-related increases in bone area, BMC and BMD were observed at the lumbar vertebra and femur. Hence, all doses of PTH(1-84) induced extensive new bone growth at multiple skeletal sites. Furthermore, the results from the toxicokinetic portion of the present study showed that the systemic exposure to PTH increased in a dose-related manner. That is, the treated rats experienced a nearly 4-fold greater exposure at the low dose (10 μg/kg/day) and a 53-fold greater exposure at the high dose (150 μg/kg/day) when compared with the exposure in humans following administration of the 100 μg clinical dose of PTH(1-84) (Table 3).

An increased incidence of osteosarcoma was observed with the mid- and the high-doses of PTH(1-84), but the incidence was lower in the females than in the males. In contrast, no bone neoplasms were observed in females receiving the low dose of PTH(1-84), whereas two osteosarcomas were observed in one of the female control groups. Soft tissue masses, which were histologically-classified as osteosarcomas, based on the identification of osteoblastic cells in those soft tissue sections, were observed in two male rats that received the low-dose of PTH(1-84). Neither mass, however, was observed to have a primary bone origin. Indeed, in one male rat, the growth was observed in the lung, while in the other rat, the mass was found in subcutaneous tissue in the ventral cervical region. The subcutaneous mass was well-encapsulated and this characteristic makes it atypical of the osteosarcomas seen in bone or in other soft tissues. However, osteosarcomas are known to occur spontaneously in subcutaneous tissues. See pages 209-226 of Boorman et al., PATHOLOGY OF THE FISCHER Rat, Leininger J R, Riley MGI (eds.) Academic Press, San Diego, Calif., 1990. Furthermore, soft tissue growths such as the growth in the subcutaneous tissue described above are known to occur, albeit rarely, in rats. The incidence of osteosarcoma in the low dose male group, excluding the spontaneous subcutaneous tumor, was 1/60(1.7%). This falls within the historical control range and is essentially equal to the mean incidence in the NTP control database. See Table 8 below.

TABLE 8
Historical control data from the National Toxicology
Program (NTP) for osteosarcoma in Fischer 344 rats

| Parameter | Males | Females |
|---|---|---|
| Range (%) | 0 to 6 | 0 to 1 |
| Mean(%) | 1.2 | 0.05 |
| Standard Deviation (%) | 1.7 | 0.23 |

Thus, treatment with the low dose, i.e., 10 μg/kg/day, of PTH(1-84) did not increase the incidence of osteosarcoma above background in either sex. Accordingly, the “No Observed Effect Level” for bone neoplasms is 10 μg/kg/day for PTH(1-84).

Recap of Results Obtained with PTH(1-84) and PTH(1-34)

The present two-year study of PTH(1-84) in rats was designed to generate results that would be comparable to those obtained from the prior, Vahle et al. supra, study of teriparatide (“the teriparatide study”). The results obtained with PTH(1-84) and with teriparatide, i.e., PTH(1-34), are similar in most respects, especially when their anabolic effects on bone are compared. However, in contrast to teriparatide, PTH(1-84) did not cause an increased incidence of osteosarcoma at clinically relevant doses.

The rats in the present study experienced exposure ratios of PTH(1-84) that were very comparable to those of the teriparatide study. For example, in the teriparatide study, the low, 5 μg/kg/day, dose resulted in a systemic teriparatide exposure about three-fold greater than that which occurred in humans prescribed a 20 μg dose. Moreover, the lowest PTH doses in both studies, i.e., 5 μg/kg/day dose of teriparatide and 10 μg/kg/day of PTH(1-84), resulted in substantial new bone growth at both axial and appendicular sites, which reflects the intended pharmacological response to parathyroid hormone treatment.

The present data indicate that PTH(1-84), when administered at a dose that results in a four fold higher exposure than that observed clinically (100 μg/dose), does not cause an increased incidence of osteosarcoma. In contrast, teriparatide, even when administered at a dose that resulted in a slightly lower exposure ratio than that obtained with PTH(1-84), increased the incidence of osteosarcoma in rats. See, Vahle et al., supra.

The two higher doses of PTH(1-84), i.e., 50 and 150 μg/kg/day did increase the incidence of osteosarcomas in rats, but the magnitude differed from that caused by the equivalent higher doses of teriparatide. Accordingly, when viewed as “exposure ratio-response curves,” this difference is readily apparent. That is, there is a shift to the right in the exposure ratio-response curve for ALX1-11 (FIG. 1). This right-ward shift correlates with a safety margin of more than four, as determined by the exposure ratio of rats to humans.

Conversely, since all•doses of teriparatide increased the incidence of osteosarcoma, no safety margin for human administration is established. In other words, any exposure to teriparatide, i.e., PTH(1-34), could increase the incidence of osteosarcoma.

Hence, analysis of the present data indicates that:

There was no difference in the incidence of osteosarcoma seen in the low-dose and control arms of the present study and/or the historical data (Table 8);

A dose-related increase in the incidence of osteosarcoma occurred in the mid- and high-dose arms of the study, but at rates that appear to be lower than those observed in published carcinogenicity studies using teriparatide; and,

The bone-building effects of PTH(1-84), as measured by increases in bone mineral density, bone mineral content, and bone size has been confirmed.

Accordingly, the present data demonstrate the dramatically superior safety profile of PTH(1-84) over conventional known PTH therapies for treatment of bone loss. The lowest dose of the only FDA-approved PTH product for use in treating bone loss, i.e., PTH(1-34), increases the incidence of osteosarcoma by 29 times. By contrast, the present invention demonstrates that treatment with the lowest dose of full length parathyroid hormone, i.e., PTH(1-84), does not increase the incidence of osteosarcoma above background in either sex.

### EXAMPLE 2

### A. Materials and Methods

To further test the hypothesis that long-term administration of PTH(1-84) would be less likely to induce osteosarcoma than teriparatide, we performed a carcinogenicity study in which rats received daily subcutaneous injections of PTH for two years.

Rats of the species Rattus norvegicus, strain Fischer 344 (F344/NHsd), obtained from Harlan Sprague Dawley (Indianapolis, Ind.) were used in the experiment. The animals were approximately 9 to 11 weeks at the onset of treatment. The animals were subject to daily subcutaneous injections of PTH (1-84) for up 104 weeks (Table 1).

Toxicokinetic analyses were conducted over the first 52 weeks.

Radiological evaluations (whole skeleton-dorsoventral and lateral planes) were conducted during the 2 weeks prior to scheduled necropsy on all surviving rats.

Complete post-mortem evaluations were conducted with comprehensive sampling of soft tissues, including macroscopic abnormalities.

Routinely sampled bones included: femur (distal left), tibia (proximal left), lumbar vertebrae (L5 and L6), sternum, and all bones with gross and radiological abnormalities.

Histological analyses were conducted using a standard carcinogenicity bioassay, with diagnostic criteria for bone proliferative changes (Vahle, J. L., et al. Toxicol. Pthol. 30:312, 2002) and independent peer review.

Ex vivo bone densitometry (BMD) was conduted using Hologic QDR-2000 plus bone densitometer (DXA) at Month 24 for right femur (proximal, central and distal) and L1-L4 vertebrae.

TABLE 9
Study Design

| | PTH Dose | Number of Animals per study phase | | | |
|---|---|---|---|---|---|
| Group No. | Level | Carcinogenicity | | Toxicokinetic^{a} | |
| Identification | (μg/kg/day) | Male | Female | Male | Female |
| 1 Vehicle Control | 0 | 60 | 60 | — | — |
| 2 Vehicle Control | 0 | 60 | 60 | 24 | 24 |
| 3 PTH - Low | 10 | 60 | 60 | 8 | 8 |
| 4 PTH - Mid | 50 | 60 | 60 | 8 | 8 |
| 5 PTH - High | 150 | 60^{b} | 60 | 24 | 24 |

^{a}Toxicokinetic animals sacrificed after 12 months of treatment.
^{b}Treatment stopped at Week 94 for Group 5 males, which remained untreated until their necropsy at Week 101.

### Statistical Analyses

Statistical comparisons vs. each control group and combined control groups were conducted as follows:
Mortality: Peto's one-sided trend test using Proc Multitest
Tumor data: Peto's survival-adjusted one-sided trend test with tumors classified as fatal or incidental using Proc Multitest
Non-neoplastic lesions: one-sided Cochran-Armitage trend test followed by Fisher's exact test for comparison between groups
DXA data: Kruskal-Wallis followed by Wilcoxon-rank-sum test or ANOVA followed by t-test, depending on result of Levene's test

### B. Results

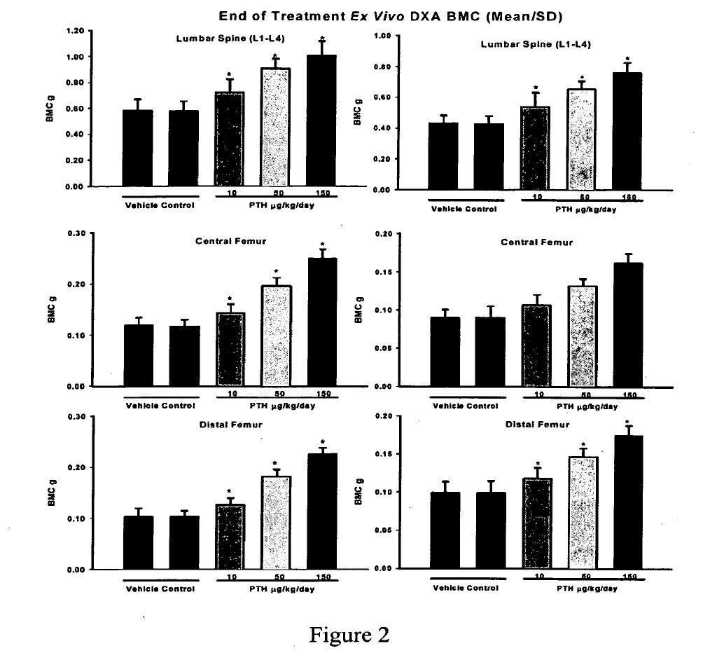

Dose-related increases in bone mineral content (BMC) were noted in lumbar spine and femur of both genders following treatment with PTH (1-84), although the increase in femur BMC was greater in males (FIG. 2). The graphs in FIG. 2 show that the mean±SD DXA BMC at the lumbar spine and central and distal regions of the femur was significantly increased for all PTH groups compared to combined controls groups after 24 months of treatment.*: p<0.001 (t-test), males and females on left and right panels, respectively.

Exposure to PTH (1-84) was dose-related in both genders and slightly lower in female rats. Steady state exposure was reflected by the 6- and 12-month measurements, which were averaged for both genders for comparison with human exposure (Table 10). Increased mortality was noted in the high dose males. An increased number of fatal osteosarcomas was seen in mid-dose males and high dose females (Table 11). The difference in mortality rate between genders was attributed primarily to a greater severity of chronic progressive nephropathy in male rats.

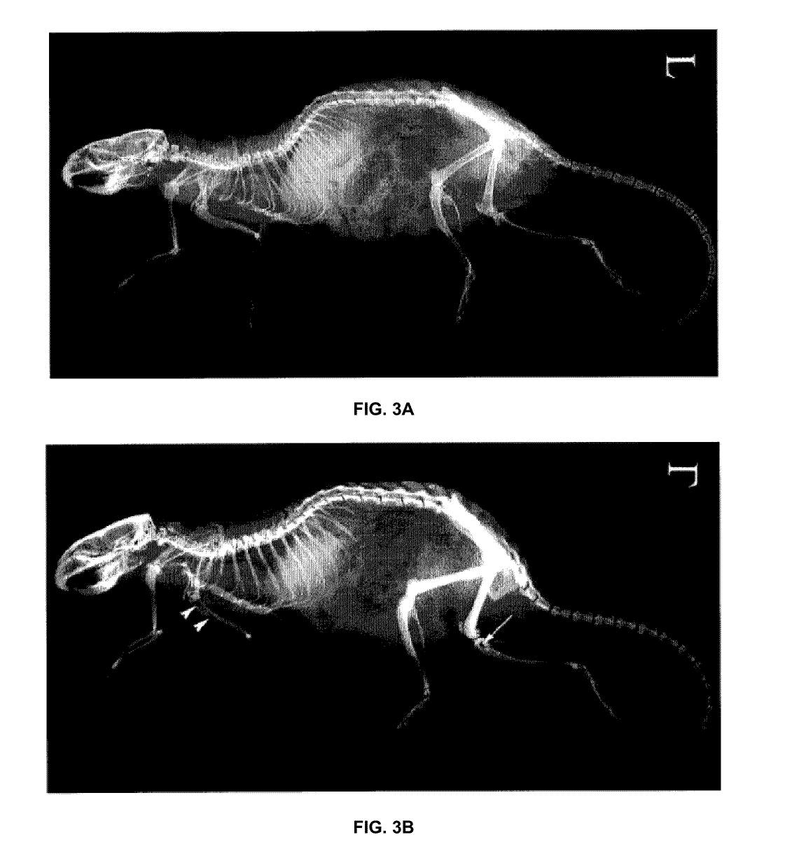

Bone sclerosis was commonly seen by x-ray in all PTH (1-84) groups (Table 11, FIGS. 3A and 3B). A control female rat is depicted in FIG. 3A and a rat treated with PTH (1-84) at 150 μg/kg/day is depicted in FIG. 3B. The rat in FIG. 3B shows a generalized increased radiodensity of the skeleton with evident thickening of the calvarium, osteosclerosis of the long bones and obliteration of medullary spaces in femurs. In addition, focal bone losses in left proximal humerus (arrowhead) and in left proximal tibia (arrow) were subsequently diagnosed as multicentric osteosarcomas. Radiography revealed a small number of tumors, which otherwise would have remained undetected in rats dosed with 50 or 150 μg/kg/day (Table 11).

TABLE 10
PTH (1-84) Exposure Ratios* at 6 and 12 months

| PTH Dose | Rat/Human Exposure Ratio | | |
|---|---|---|---|
| (μg/kg/day) | Month 6 | Month 12 | Mean |
| 10 | 4.6 | 4.7 | 4.6 |
| 50 | 28 | 26 | 27 |
| 150 | 60 | 72 | 66 |

*Exposure ratios = AUCᵣₐₜ/AUCₕᵤₘₐₙ; where AUCₕᵤₘₐₙ = 0.8 ng hr/mL in postmenopausal osteoporotic women receiving 100 μg dose (Phase III). A two-site IRMA (Scantibodies, Inc.) with high specificity for full-length PTH was used to quantitate human PTH levels in both species.

TABLE 11
Mortality and radiology data

| | Sex | | | | | | | | | |
|---|---|---|---|---|---|---|---|---|---|---|
| | Male | | | | | Female | | | | |
| | PTH Dose (μg/kg/day) | | | | | | | | | |
| | 0 | 0 | 10 | 50 | 150 | 0 | 0 | 10 | 50 | 150 |
| No. of Animals | 60 | 60 | 60 | 60 | 60 | 60 | 60 | 60 | 60 | 60 |
| Mortality: | 42 | 44 | 44 | 44 | 46^{#} | 25 | 29 | 33 | 39 | 35 |
| No. of animals | | | | | | | | | | |
| Fatal | 0 | 0 | 1 | 7 | 10 | 1 | 0 | 0 | 2 | 8 |
| Osteosarcoma: | | | | | | | | | | |
| No. of Animals | | | | | | | | | | |
| Subjected to | 25 | 18 | 21 | 19 | 16 | 36 | 34 | 28 | 24 | 25 |
| X-Ray: | | | | | | | | | | |
| No. of Animals | | | | | | | | | | |
| Bone Sclerosis: % | 4 | 0 | 38 | 95 | 100 | 3 | 9 | 25 | 100 | 100 |
| No. of Occult | 0 | 0 | 0 | 1 | 0 | 0 | 0 | 0 | 1 | 4 |
| Bone | | | | | | | | | | |
| Neoplasms^{&} | | | | | | | | | | |

^{#}Different from combined control groups at Week 94 (p < 0.05, Peto's test)
^{&}Histologically-confirmed primary bone neoplasms where radiography was essential for diagnosis; 5 osteosarcomas and 1 osteoblastoma

### Pathological Evaluation

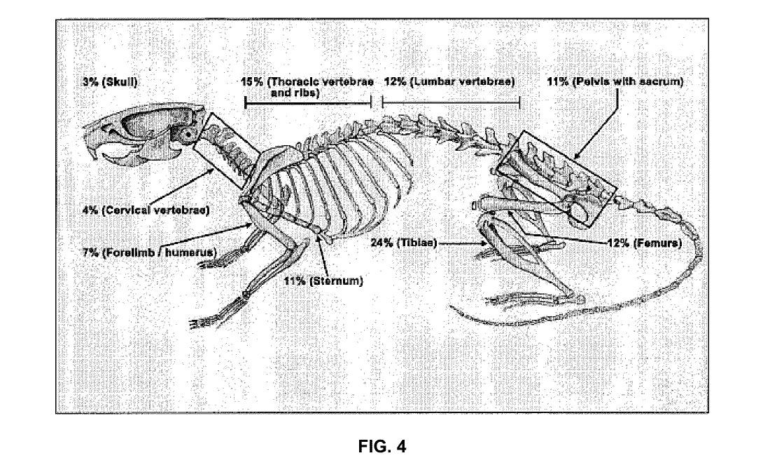

A spectrum of bone neoplasms occurred in all groups of rats treated with PTH (1-84) at 50 or 150 μg/kg/day (Table 12) in both the appendicular and axial skeleton (FIG. 4). Metastases were noted in 45% of all PTH (1-84)-treated rats diagnosed with ostesarcoma.

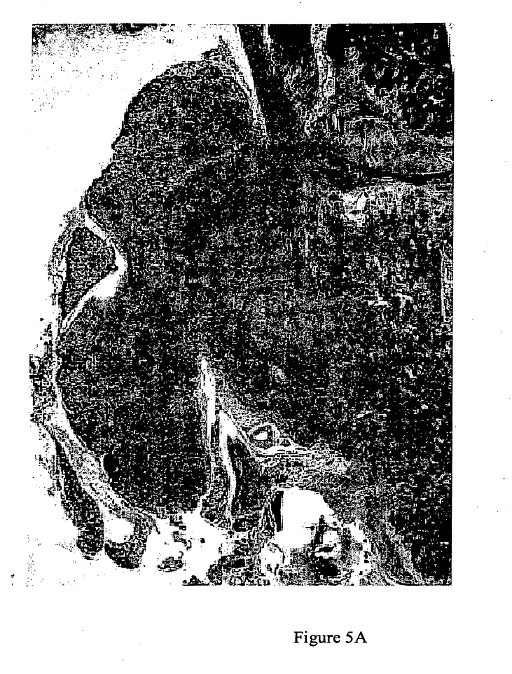

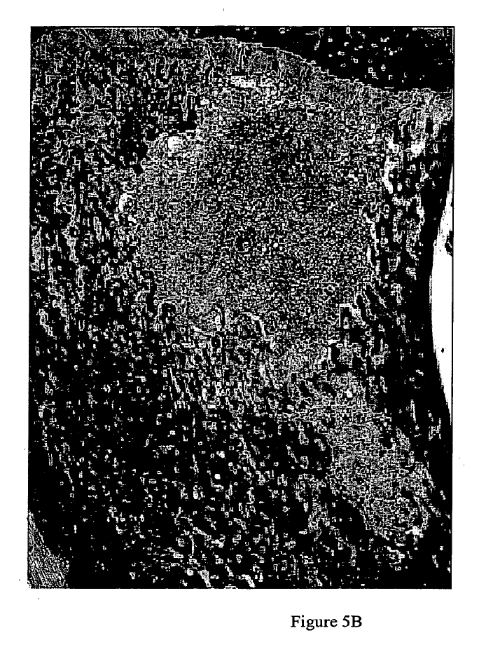

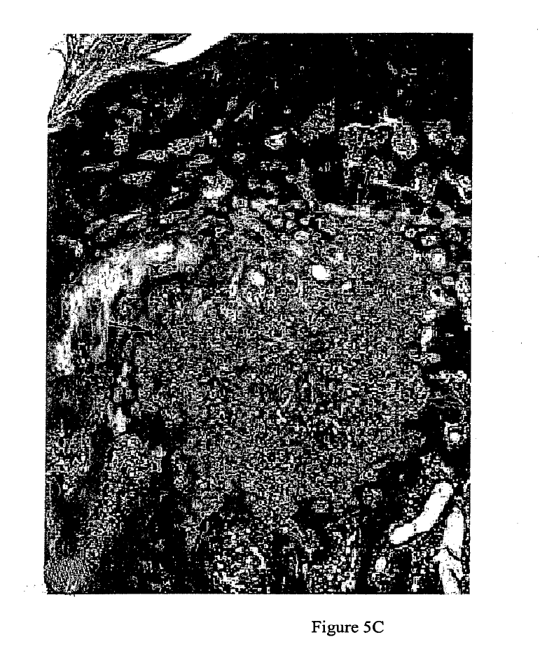

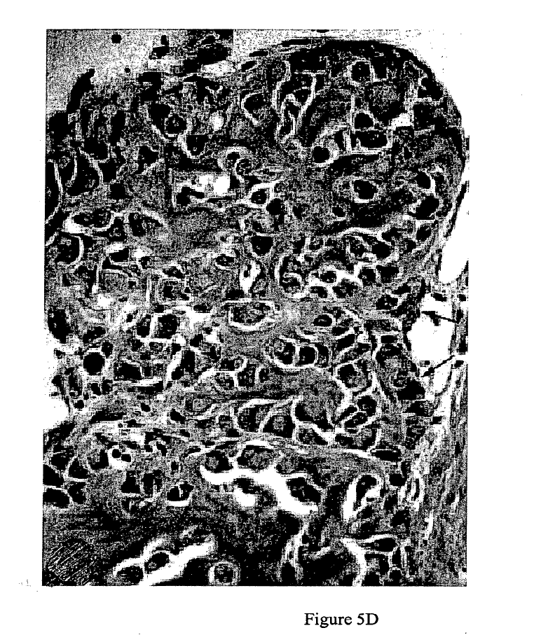

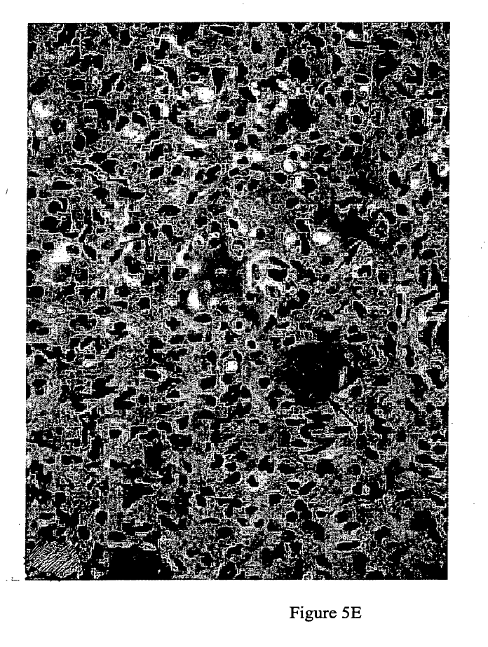

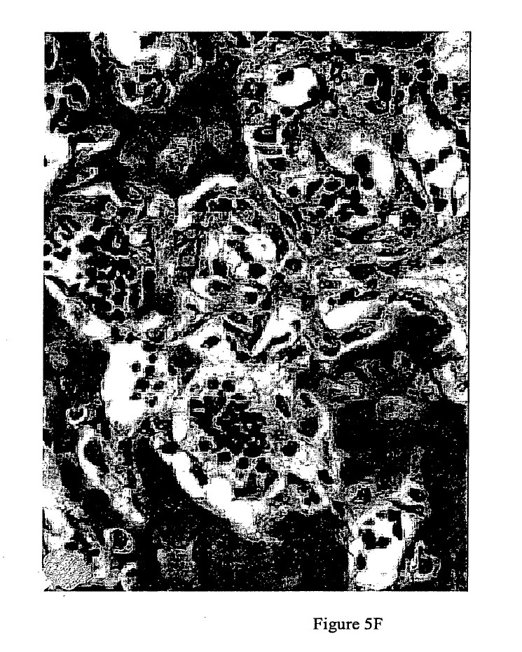

The histological pattern of osteosarcomas was variable; the osteoplastic subtype was most frequent (FIGS. 5A-5F). FIGS. 5A and 5D depict a female rat given 150 μg/kg/day PTH (1-84). Osteoplastic osteosarcoma was observed in thoracic vertebral body. The zonation phenomenon is evident with centrally located tumor bone(*) and anapestic osteopblasts at outer margin (arrow). FIGS. 5B and 5E depict a female rat given 150 μg/kg/day PTH (1-84). Fibroblastic osteosarcoma was observed in tibial proximal metaphysic (arrowhead). Radiographic evaluation, shown in FIG. 3B, was required to diagnose this neoplasm. FIGS. 5C and 5F depict a male rat given 50 μg/kg/day PTH (1-84). Well defined osteoblastoma (arrows) were observed in tibial proximal metaphysis.

Other bone neoplasms regarded as PTH (1-84)-related included benign osteoblastoma (FIG. 4) and osteoma, and malignant fibrosarcoma (Table 12). There was no increased incidence of bone neoplasia in rats dosed with PTH (1-84) at 10 μg/kg/day as compared with controls (Table 12). Both groups had an incidence comparable to that reported in control Fischer 344 rats by the National Toxicology Program (NTP). Treatment with PTH (1-84) did not induce neoplasia in any other tissues.

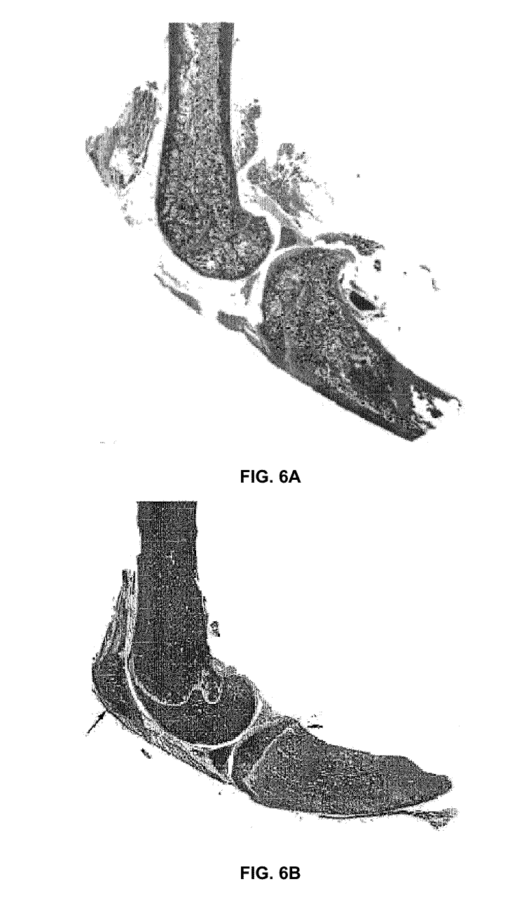

Focal osteoblast hyperplasia, occurring primarily in the tibia, lumbar vertebra and femur, was enhanced only by the mid and high doses of PTH (1-84) (Table 13). In the tested rats, osteosclerosis was widespread across all bones in both genders at all PTH (1-84) dose levels (Table 13, FIGS. 6A and 6B). FIG. 6A shows a control female. FIG. 6B shows a female treated with 150 μg/kg/day PTH (1-84). Severe diffuse osteosclerosis with complete obliteration of the marrow cavity was observed in femur, tibia and patella (arrow).

Other non-neoplastic histological findings in bones associated with PTH(1-84) doses of 50 or 150 μg/kg/day included: a low incidence of fibrous osteodystrophy (unrelated to age-related nephropathy) in both genders and occasional occurrences of osteofibrous dysplasia in females.

TABLE 12
Noteworthy neoplasms found in bones*

| | Sex | | | | | | | | | |
|---|---|---|---|---|---|---|---|---|---|---|
| | Male | | | | | Female | | | | |
| | PTH dose (μg/kg/day) | | | | | | | | | |
| | 0 | 0 | 10 | 50 | 150 | 0 | 0 | 10 | 50 | 150 |
| Number of | 60 | 60 | 60 | 60 | 60 | 60 | 60 | 60 | 60 | 60 |
| animals | | | | | | | | | | |
| No. of animals | | | | | | | | | | |
| affected with | | | | | | | | | | |
| one or more: | | | | | | | | | | |
| Osteoblastoma | 0 | 0 | 0 | 2 | 4* | 0 | 0 | 0 | 3* | 9* |
| Osteoma | 0 | 0 | 0 | 1 | 2* | 0 | 0 | 0 | 1 | 1 |
| Osteosarcoma | 0 | 0 | 1 | 13* | 27* | 2 | 0 | 0 | 5 | 13* |
| Fibrosarcoma | 0 | 0 | 0 | 1 | 0 | 0 | 0 | 0 | 1 | 0 |
| All bone | 0 | 0 | 1 | 17* | 30* | 2 | 0 | 0 | 10* | 21* |
| neoplasms | | | | | | | | | | |
| Multicentric | 0 | 0 | 0 | 0 | 10 | 0 | 0 | 0 | 1 | 4 |
| neoplasm^{&} | | | | | | | | | | |
| Metastatic | 0 | 0 | 1 | 6 | 14 | 1 | 0 | 0 | 1 | 7 |
| osteosarcoma | | | | | | | | | | |

^{#} All primary neoplasms, including metastatic osteosarcomas found in soft tissues of 5 preterminal rats and considered to originate from an undetermined skeletal origin
^{&}Combination of above listed tumors and/or multicentric osteosarcomas
*Different from combined control groups (p < 0.05, Peto's test)

TABLE 13
Relevant non-neoplastic histological findings in bones

| | Sex | | | | | | | | | |
|---|---|---|---|---|---|---|---|---|---|---|
| | Male | | | | | Female | | | | |
| | PTH dose (μg/kg/day) | | | | | | | | | |
| | 0 | 0 | 10 | 50 | 150 | 0 | 0 | 10 | 50 | 150 |
| Number of animals | 60 | 60 | 60 | 60 | 60 | 60 | 60 | 60 | 60 | 60 |
| Finding/Tissue | | | | | | | | | | |
| Focal osteoblast | 1 | 2 | 0 | 5 | 12* | 1 | 3 | 1 | 4 | 12* |
| hyperplasia/All sites^{#} | | | | | | | | | | |
| Osteosclerosis/ | | | | | | | | | | |
| Femur | 1 | 0 | 26* | 58* | 60* | 14 | 12 | 44* | 60* | 60* |
| Lumbar vertebra | 2 | 2 | 14* | 51* | 60* | 11 | 10 | 43* | 59* | 60* |
| Sternum | 0 | 2 | 10* | 57* | 60* | 18 | 13 | 38* | 59* | 60* |
| Tibia | 0 | 0 | 15* | 58* | 60* | 11 | 11 | 41* | 60* | 60* |

^{#}From all bones examined
*Different from controls (p < 0.05, Fisher's exact test)

### C. Role of C-Terminal Region of PTH (1-84)

The N-terminal region of PTH (1-84) activates the PTH-IR, which regulates calcium homeostasis and bone turnover. C-terminal fragments of PTH (1-84), such as PTH(39-84), neither bind to nor activate PTH-1R. This led to the conclusion that the C-terminal region of PTH is biologically inactive (Potts J T, Juppner H. In: Metabolic Bone Disease. Academic Press 1998, p51; and Gardella T J, et al. In: Principles of Bone Biology. Academic Press 2002, p389).

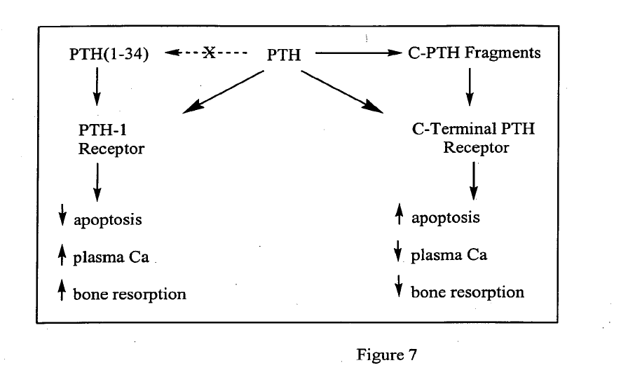

However, it has now been shown that bone cells express a distinct PTH receptor, which responds only to the C-terminal region of PTH (Rao L G, Murray T M. Endocrinology 117:1632, 1985; and Inomata N, et al. Endocrinology 136:4732, 1995). Numerous in vitro and in vivo studies have demonstrated that the N- and C-terminal regions of PTH have opposing effects on alkaline phosphatase activity and apoptosis in bone cells, bone resorption and turnover, and on plasma calcium levels (Murray T M, et al. Endocrinology 124:1097, 1989; Divieti P, et al. Endocrinology 142:916, 2001; Jilka R L, et al. J Clin Invest 104:439, 1999; Divieti P, et al. Endocrinology 143:171, 2002; Langub M C, et al. Endocrinology 144:1135, 2003; Nguygen-Yamamoto L, et al. Endocrinology 142:1386, 2001; and Slatopolsky E, et al. Kidney Int 58:753, 2000). (FIG. 7)

Metabolism of plasma PTH (1-84) occurs primarily within hepatic Kupffer cells. C-terminal fragments are returned to the circulation while the N-terminal region is degraded in situ (Segre G V, et al. J Clin Invest 67:449, 1981; Hruska K A, et al. J Clin Invest 67:885, 1981; Goltzmann D, et al. Recent Prog Horm Res 42:665, 1986; and Bringhurst F R, et al. Am J Physiol 255:E886,1988). This provides a regulatory mechanism whereby the N-terminal region of PTH (1-84) activates a physiological response, which is followed by a C-terminal fragment-mediated counter response.

An anabolic agent for the treatment of osteoporosis will induce new bone growth without overstimulating the PTH-1R, which may lead to neoplasia. A distinct receptor that recognizes the C-terminal region of PTH (but not teriparatide) has been described, which may provide such a regulatory mechanism.

### D. Discussion

Histological and densitometry end-points confirmed significant new bone growth with long-term PTH administration at 10 μg/kg, but without the neoplastic complications observed at higher doses.

The assurance that a no-carcinogenic effect dose level was found with 10 μg/kg PTH was supported by radiological examination, which allowed detection of skeletal lesions that would otherwise have remained occult at necropsy. In addition, compared to routine oncogenicity studies, the histopathological evaluation of the skeleton was more thorough in the present study.

The C-terminal region of PTH is responsible for the lower incidence of osteosarcoma in this study than previously reported for teriparatide, which only activates the PTH-1R (Potts J T, Juppner H. In: Metabolic Bone Disease. Academic Press 1998, p51 and Gardella T J, et al. In: Principles of Bone Biology. Academic Press 2002, p389).

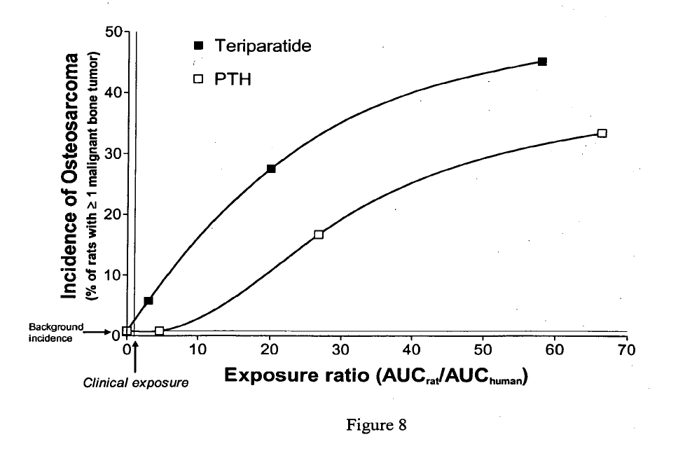

The right-shift in the PH dose-response curve for osteosarcoma, relative to teriparatide, affords a margin of safety for PTH of at least 4.6-fold between the human clinical dose and the no-carcinogenic dose in rats (FIG. 8).

### E. Summary and Conclusions

In the tested rats, all doses of PTH induced significant new bone growth at all skeletal sites evaluated.

There was no increase in the incidence of proliferative, benign or malignant neoplastic bone changes in the 10 μg/kg/day dose group as compared to control rats.

The non-carcinogenic PTH dose was 10 μg/kg.

Increased proliferative and neoplastic lesions, primarily osteosarcoma, were observed at PTH doses ≧50 μg/kg.

The incidence of osteosarcoma was lower at all PTH doses when compared to teriparatide at similar systemic exposures.

The results of this study support the hypothesis that the C-terminal region of PTH modulates the proliferative effects on cells of the osteoblastic lineage induced by PTH-1R activation.

It will be apparent to those skilled in the art that various modifications and variations can be made in the methods and compositions of the present invention without departing from the spirit or scope of the invention. Thus, it is intended that the present invention cover the modifications and variations of this invention provided they come within the scope of the appended claims and their equivalents.

What is claimed is:

1. A method for treating a subject suffering from, or at risk of, bone loss comprising administering to said subject a composition comprising a dosage of full-length parathyroid hormone (PTH(1-84)), wherein said dosage does not exceed about 600 μg/person/day, wherein said subject is selected from a patient population suffering from, or at risk of, developing osteosarcoma.

2. A method for treating a subject suffering from, or at risk of, bone loss comprising administering to said subject a composition comprising a dosage of full-length parathyroid hormone (PTH(1-84)), wherein said dosage does not exceed about 600 μg/person/day, wherein said subject is selected from a patient population which do not take PTH(1-34) because of the increased risk of osteosarcoma associated with PTH(1-34) in rats.

3. The method of claim 1, wherein the dosage does not exceed about 400 μg/person/day.

4. The method of claim 1, wherein the dosage does not exceed about 370 μg/person/day.

5. The method of claim 1, wherein the dosage does not exceed about 200 μg/person/day.

6. The method of claim 1, wherein the dosage is from about 25 μg/person/day to about 150 μg/person/day.

7. The method of claim 6, wherein the PTH(1-84) comprises the amino acid sequence of SEQ ID NO. 1.

8. The method of claim 6, wherein said patient population is selected from the group consisting of: (1) subjects with Paget's disease of bone; (2) subjects with elevated levels of alkaline phosphatase; (3) pediatric subjects or young adults with open epiphyses; (4) subjects having a prior history of radiation therapy involving the skeleton; (5) subjects with bone metastases; 6) subjects having a history of skeletal malignancies; (6) subjects with metabolic bone diseases other than osteoporosis; and (7) subjects having any combination of (1) through (6).

9. The method of claim 6, wherein said subject is suffering from, or at risk of, bone loss due to osteoporosis.

10. The method of claim 9 wherein said subject is suffering from bone loss due to osteoporosis.

11. The method of claim 10, wherein the parathyroid hormone is human parathyroid hormone.

12. The method of claim 11, wherein the composition is administered daily.

13. The method of claim 12, wherein the composition is administered over a period not to exceed two (2) years.

14. The method of claim 12, wherein the composition is in lyophilized form.

15. The method of claim 14, wherein the composition is reconstituted with water into a liquid form.

16. The method of claim 15, wherein the composition is administered to the subject by injection.

17. The method of claim 13, wherein the subject is a postmenopausal female and the dosage of the parathyroid hormone in the pharmaceutical composition is about 100 μg/per subject per day.

18. The method according to claim 10, further comprising administering a compound selected from the group consisting of vitamin D, dietary calcium, bisphosphonates, alendronate, estrogen, selective estrogen receptor modulators, raloxifene, tamoxitene, droloxifene, calcitonin, R1, BIS, GM-CSF, hGRF(144)-NH₂, benzo-fused lactam, 5-AR-I inhibitors, benzoquinolin-3-ones, VEGF, GLP-2, and combinations thereof.

19. The method according to claim 17, wherein said compound is selected from the group consisting of calcium, vitamin D, bisphosphonates, alendronate, raloxifene, and combinations thereof.

20. A method for reducing the risk of osteosarcoma of an N-terminal PTH fragment in a subject, comprising administering to the subject a pharmaceutical composition comprising intact parathyroid hormone (PTH(1-84)).

21. A method for reducing the risk of osteosarcoma induced by an N-terminal PTH fragment in a subject, comprising administering to the subject a pharmaceutical composition comprising a C-terminal fragment of parathyroid hormone.

22. A method for treating a subject suffering from, or at risk of, bone loss comprising administering to the subject a pharmaceutical composition comprising full-length parathyroid hormone (PTH (1-84), wherein: (a) such administration does not result in a significant increased risk of osteosarcoma; and (b) the subject is selected from a patient population which do not take PTH(1-34) because of the increased risk of osteosarcoma associated with PTH(1-34) in rats.

23. The method according to claim 21 wherein said subject is suffering from osteoporosis.

24. A method for treating a subject suffering from, or at risk of, bone loss comprising administering to the subject a pharmaceutical composition comprising full-length parathyroid hormone (PTH(1-84)) wherein:
(a) such administration does not result in a significant increased risk of osteosarcoma; and
(b) the subject is selected from the group consisting of: (1) subjects with Paget's disease of bone; (2) subjects with elevated levels of alkaline phosphatase; (3) pediatric subjects or young adults with open epiphyses; (4) subjects having a prior history of radiation therapy involving the skeleton; (5) subjects with bone metastases or a history of skeletal malignancies;
and (6) subjects with metabolic bone diseases other than osteoporosis.

25. The method according to claim 23 wherein said subject is suffering from osteoporosis.

26. A package comprising one or more daily doses of a 100 μg/day dose of full-length PTH(1-84) and written instructions wherein said written instructions provide that said pharmaceutical composition is to be administered to a patient who is suffering from osteoporosis and wherein risk of increased osteosarcoma has not been seen in rats at dosages exhibiting about a 3 to 4 fold increase in exposure to PTH(1-84).

27. A package according to claim 26 wherein there are fourteen (14) daily doses of a 100 μg/day dose of full-length PTH(1-84).

28. The package according to claim 26 wherein said written instructions provide that the risk of increased osteosarcoma has not been seen in rats at dosages exhibiting nearly a 4 fold increase in exposure to PTH(1-84).

29. The package according to claim 26 wherein said written instructions provide that the risk of increased osteosarcoma has not been seen in rats at dosages exhibiting a 3 fold increase in exposure to PTH(1-84).
